(19) United States
(12) Patent Application Publication
(10) Pub. No.: US 2026/0055209 A1
(43) Pub. Date: Feb. 26, 2026

(54) Nanobody Target GCC and Uses in Chimeric Antigen Receptor Cell Therapy

(71) Applicants: Innovative Cellular Therapeutics Holdings, Ltd., George Town (KY); Innovative Cellular Therapeutics, Inc., Rockville, MD (US)

(72) Inventors: Zhiyuan Cao, Rockville, MD (US); Wei Ding, Shanghai (CN); Guiting Han, Shanghai (CN); Yuzhe Peng, Shanghai (CN); Lei Xiao, Rockville, MD (US); Zhao Wu, Rockville, MD (US); Le Tian, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 19/105,586

(22) PCT Filed: Aug. 22, 2023
(86) PCT No.: PCT/US2023/030846

§ 371 Date: Feb. 21, 2025

Related U.S. Application Data
(63) Continuation of application No. 17/821,706, filed on Aug. 23, 2022, now Pat. No. 11,970,548.

(51) Int. Cl. C07K 16/40; A61K 38/00; A61K 40/11; A61K 40/31; A61K 40/42; A61P 35/00; C07K 14/725

(52) U.S. Cl. CPC C07K 16/40; A61K 40/11; A61K 40/31; A61K 40/4244; A61P 35/00; C07K 14/7051; A61K 38/00; C07K 2317/56; C12Y 406/01002

(57) ABSTRACT

The present disclosure describes compositions and methods of treating solid tumors using chimeric antigen receptor (CAR) T cell (CAR-T) therapy and/or antibodies targeting uanylate Cyclase 2C (GCC. GUCY2C). The compositions include CAR comprising an extracellular domain that binds GCC. Further disclosed are methods of using modified T ceils expressing a CAR that binds GCC for treating different types of carters.

### CROSS REFERENCE TO RELATED APPLICATIONS

This application claims the benefit of U.S. application Ser. No. 17/821,706, filed Aug. 23, 2022, which is hereby incorporated by reference in its entirety.

### SEQUENCE LISTING INFORMATION

A computer-readable XML file, entitled “I071-0098EP.xml” was created on or about Dec. 15, 2022, 2022 with a file size of about 492,329 bytes, contains the sequence listing for this application and is hereby incorporated by reference in its entirety.

### TECHNICAL FIELD

The present disclosure relates to modified cells and uses, in particular to compositions and methods for treating cancer using Chimeric Antigen Receptor (CAR) cells.

### BACKGROUND

A heavy chain (VHH) antibody (or nanobody) is the antigen binding fragment of heavy chain only antibodies. It has been reported that single VHH directed BCMA CAR T cells caused remission of relapsed/refractory multiple myeloma. Therefore, VHH antibodies can be used to direct CAR T cells to target solid tumors.

### SUMMARY

Embodiments relate to a chimeric antigen receptor (CAR) comprising a transmembrane domain, an intracellular domain, and an extracellular domain, and the CAR comprises any one of SEQ ID NOs: 237, 225, and 471-476.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram illustrating an exemplary CAR molecule.

FIG. 2 shows an exemplary structure of a binding molecule.

FIG. 3 shows an exemplary structure of a CAR molecule.

FIG. 4 shows flow cytometry results of antibody binding assay.

FIG. 5 shows flow cytometry results of antibody binding assay.

FIG. 6 shows flow cytometry results of antibody binding assay.

FIGS. 7A, 7B, and 7C show GCC CAR expression and activation 7 days after the transduction.

FIGS. 8A, 8B, and 8C show the activation of GCC CAR T cells after co-culturing with GCC-positive cells.

FIGS. 9A, 9B, and 9C show the exhaustion of GCC CAR T cells after co-culturing with GCC-positive cells for 24 hours.

FIGS. 10A, 10B, and 10C show the phenotypes (memory/effector) of GCC CAR T cells after co-culturing with GCC-positive cells.

FIG. 11 shows the results of a killing assay of GCC CAR T cells after co-culturing with GCC-positive cells.

FIG. 12 shows the release of cytokines after GCC CAR co-cultivation.

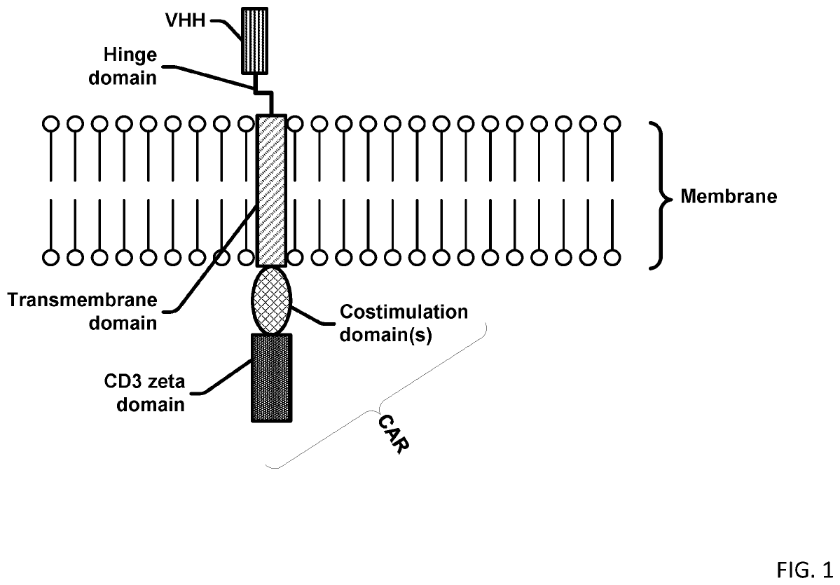

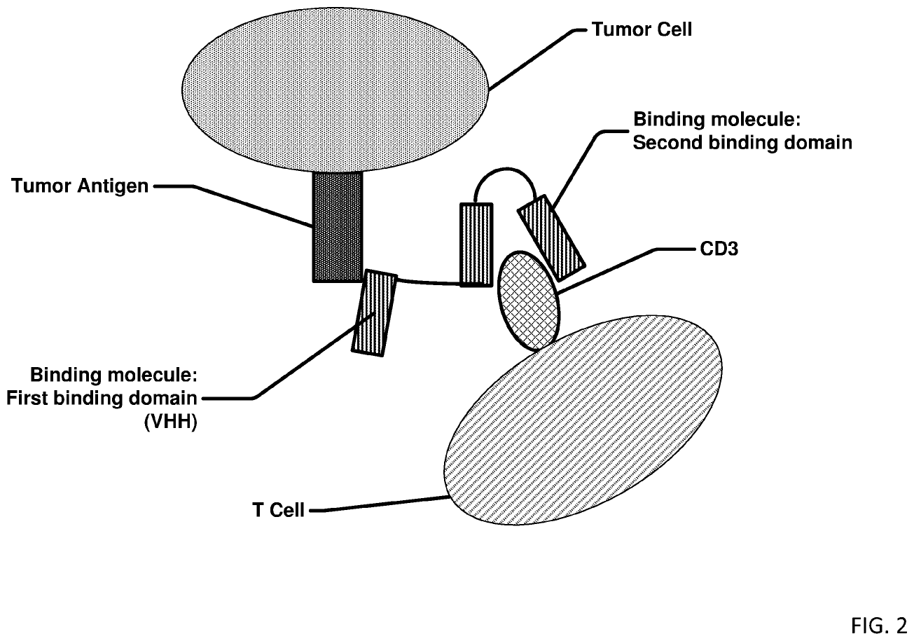

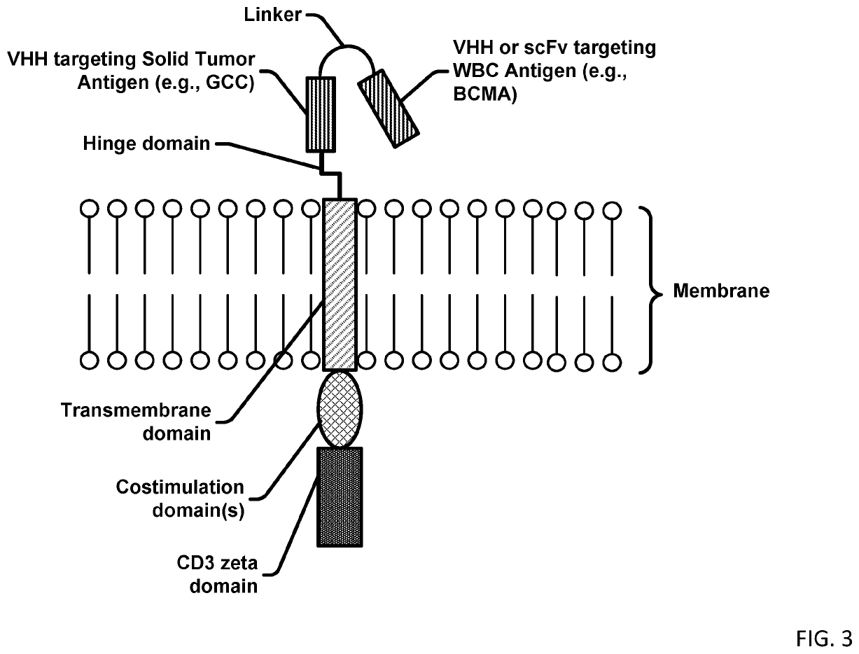

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, and Fv, Fab, Fab' and F(ab')₂ and fragments, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab', F(ab')₂, and Fv fragments; diabodies; linear antibodies; single-chain antibody molecules; and multi-specific antibodies formed from antibody fragments.

The term “Fv” refers to the minimum antibody fragment which contains a complete antigen-recognition and-binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “autoantigen” or “self-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer, and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| (IL-13 receptor | |
| alpha 2) | |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, Pancreatic cancer, |
| PSCA | pancreas, stomach, or prostate cancer |

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements or steps are required or mandatory and that no other elements may be present.

The phrase “consisting essentially of” is meant to include any element listed after the phrase and can include other elements or steps that do not interfere with or contribute to the activity or action specified in the disclosure for the listed elements or steps. Thus, the phrase “consisting essentially of” indicates that the listed elements or steps are required or mandatory, but that other elements or steps are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring, for example, to CD3 zeta, the primary signaling domain. In embodiments, the co-stimulatory signaling domain and the stimulatory (or primary) signaling domain can be on the same molecule or different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter. The term “overexpression” refers to the production of a gene product in transgenic organisms or cells that exceeds levels of production in normal or non-transformed organisms or cells.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (AAV)) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared x100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein, and refer to any animal, such as a mammal, for example, a human or any living organism amenable to the methods described herein. In embodiments, the patient, subject, or individual is a human or mammal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. Accordingly, the subject can also be in need of prevention of a disease condition or disorder. In embodiments, the disease is cancer.

The term “polynucleotide” or “nucleic acid” refers to mRNA, RNA, CRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In embodiments, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“NFAT promoter” refers to one or more NFAT binding sites or motifs linked to a minimal promoter of any gene expressed by T cells. In embodiments, the minimal promoter of a gene expressed by T cells is a minimal human IL-12 promoter. NFAT (nuclear factor of activated T cells) are transcription factors. Examples of NFAT transcription factors include NFAT1, NFAT2, NFAT3, NFAT4, and NFAT5. These transcription factors bind NFAT binding sites or motifs in the NFAT promoter. The NFAT promoter (or a functional portion or functional variant thereof) can comprise any number of binding motifs, e.g., at least two, at least three, at least four, at least five, or at least six, at least seven, at least eight, at least nine, at least ten, at least eleven, or up to twelve binding motifs. In embodiments, the NFAT promoter comprises six NFAT binding motifs. In an especially preferred embodiment, the NFAT promoter nucleotide sequence comprises or consists of a functional portion or functional variant thereof.

The NFAT promoter (or a functional portion or functional variant thereof) is operatively associated with the nucleotide sequence encoding IL-12 (or a functional portion or functional variant thereof). “Operatively associated with” means that the nucleotide sequence encoding IL-12 (or a functional portion or functional variant thereof) is transcribed into IL-12 mRNA when the NFAT protein binds to the NFAT promoter sequence (or a functional portion or functional variant thereof). Without being bound to a particular theory, it is believed that NFAT is regulated by a calcium signaling pathway. In particular, it is believed that TCR stimulation (by, e.g., an antigen) and/or stimulation of the calcium signaling pathway of the cell (by, e.g., PMA/lonomycin) increases intracellular calcium concentration and activates calcium channels. It is believed that the NFAT protein is then dephosporylated by calmoduin and translocates to the nucleus where it binds the NFAT promoter sequence (or a functional portion or functional variant thereof) and activates downstream gene expression. By providing an NFAT promoter (or a functional portion or functional variant thereof) that is operatively associated with the nucleotide sequence encoding IL-12 (or a functional portion or functional variant thereof), the nucleic acids described herein advantageously make it possible to express IL-12 (or a functional portion or functional variant thereof) only when the host cell including the nucleic acid is stimulated by, e.g., PMA/lonomycin and/or an antigen. More information can be found at U.S. Pat. No. 8,556,882, which is incorporated by the reference.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of statistical significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcRy were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15;90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (, “A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl. 1, May 2014, page S57) tested a CAR molecule in which an scFv was fused to “the transmembrane and cytoplasmic domain of” a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3˜-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 Jul. 3 (7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell. The cell can be an in vitro cell or a in vivo cell in a subject. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

In embodiments, a polynucleotide encoding the antigen binding molecule and/or therapeutic agent(s) can be used to implement techniques described herein. The method or use includes: providing a viral particle (e.g., AAV, lentivirus or their variants) comprising a vector genome, the vector genome comprising the polynucleotide, wherein the polynucleotide is operably linked to an expression control element conferring transcription of the polynucleotide; and administering an amount of the viral particle to the subject such that the polynucleotide is expressed in the subject. In embodiments, the AAV preparation may include AAV vector particles, empty capsids and host cell impurities, thereby providing an AAV product substantially free of AAV empty capsids. More information of the administration and preparation of the viral particle may be found at the U.S. Pat. No. 9,840,719 and Milani et al., Sci. Transl. Med. 11, eaav7325 (2019) 22 May 2019, which are incorporated herein by reference. In embodiments, the polynucleotide may integrate into the genome of the modified cell and the progeny of the modified cell will also express the polynucleotide, resulting in a stably transfected modified cell. In embodiments, the modified cell expresses the polynucleotide encoding the CAR but the polynucleotide does not integrate into the genome of the modified cell such that the modified cell expresses the transiently transfected polynucleotide for a finite period of time (e.g., several days), after which the polynucleotide is lost through cell division or other factors. For example, the polynucleotide is present in the modified cell in a recombinant DNA construct, in an mRNA, or in a viral vector, and/or the polynucleotide is an mRNA, which is not integrated into the genome of the modified cell.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The T cell response in a subject refers to cell-mediated immunity associated with a helper, killer, regulatory, and other types of T cells. For example, T cell response may include activities such as assistance to other white blood cells in immunologic processes and identifying and destroying virus-infected cells and tumor cells. T cell response in the subject may be measured via various indicators such as the number of virus-infected cells and/or tumor cells that T cells kill, an amount of cytokines that T cells release, for example, in co-culturing with virus-infected cells and/or tumor cells, a level of proliferation of T cells in the subject, a phenotype change of T cells (e.g., changes to memory T cells), and the longevity or lifespan of T cells in the subject.

In embodiments, in vitro killing assay may be performed by measuring the killing efficacy of CAR T cells by co-culturing CAR T cells with antigen-positive cells. CAR T cells may be considered to have killing effect on the corresponding antigen-positive cells by showing a decrease in the number of corresponding antigen-positive cells co-cultured with CAR T cells and an increase in the release of cytokines such as IFN-γ, TNF-α, and the like, as compared to control cells that do not express the corresponding antigen. Further, in vivo antitumor activity of the CAR T cells may be tested. For example, xenograft models can be established using the antigens described herein in immunodeficient mice. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2,-247-250 (2007)). To evaluate the anti-tumor activity of CAR T cells in vivo, immunodeficient mice bearing tumor xenografts were evaluated for CAR T cell anti-tumor activity, for example, a decrease in mouse tumors and/or mouse blood cytokines, such as IFN-γ, TNF-α, and the like.

The term “chimeric antigen receptor” or alternatively a “CAR” refers to a recombinant polypeptide construct comprising at least an extracellular antigen binding domain, a transmembrane domain, and an intracellular domain (cytoplasmic domain) including an intracellular signaling domain. In embodiments, the domains in the CAR polypeptide are on the same polypeptide chain (for example, comprising a chimeric fusion protein). In embodiments, the domains of the CAR polypeptide are not on the same molecule, for example, not contiguous with each other, or are on different polypeptide chains.

In embodiments, the intracellular signaling domain can include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there can be incorporated a spacer domain. As used herein, the term “spacer domain” generally means any oligo-or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (for example, a scFv, a single domain antibody, or TCR, such as a TCR alpha binding domain or a TCR beta binding domain), that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19 CAR (19CAR, CD19CAR, CD19 CAR, or CD19-CAR), which is a CAR molecule that includes an antigen binding domain that binds CD19.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID: 90), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6,IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR includes any antigen binding moiety that when bound to its cognate antigen, affects a tumor cell such that the tumor cell fails to grow, decrease in size, or dies.

The CAR can be a bispecific CAR. For example, the two antigen binding domains are on the same CAR (a bispecific CAR or tandem CAR (tanCAR)), on different CAR molecules, or on a CAR and T cell receptor (TCR). A single CAR can include two different antigen binding domains, or the two different antigen binding domains are each on a separate CAR. The CAR can have more than two antigen binding domains, for example, a multispecific CAR. The antigen binding domains of the multispecific CAR can be on the same CAR or on separate CAR molecules, such as one antigen binding domain on each CAR.

In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+ T lymphocytes and CD8+ T lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells may be derived from a healthy donor, from a patient diagnosed with cancer, or from a patient diagnosed with an infection. In embodiments, the cells are part of a mixed population of cells which present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types of stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells-with the capacity of fetal stem cells being greater than that of adult stem cells; they apparently differentiate into only a limited number of different types of cells and have been described as multipotent. “Tissue-specific” stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (IPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem(ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin, and blood cells.

In embodiments, the CAR cells, the modified cell, or the cell is a T cell, a NK cell, a macrophage or a dendritic cell. For example, the CAR cells, the modified cell, or the cell is a T cell.

T cells, or T lymphocytes, are a type of white blood cell of the immune system. There are various types of T cells including T helper (TH) cells, cytotoxic T (TC) cells (T killer cells, killer T cells), natural killer T (NKT) cells, memory T (Tm) cells, regulatory T (Treg) cells, and gamma delta T (γδ T) cells.

T helper (TH) cells assist other lymphocytes, for example, activating cytotoxic T cells and macrophages and maturation of B cells into plasma cells and memory B cells. These T helper cells express CD4 glycoprotein on their surface and are also known as CD4+ T cells. Once activated, these T cells divide rapidly and secrete cytokines.

Cytotoxic T (TC) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (TCM) cells, effector memory T (TEM) cells, tissue resident memory T (TRM) cells, and virtual memory T cells. Tm cells are either CD4+ or CD8+ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the αβ TCR chains. γδ T cells are less common in human and mice and are mainly found in the gut mucosa, skin, lung, and uterus. They are involved in the initiation and propagation of immune responses.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains.

In embodiments, a T cell clone that expresses a TCR with high affinity for the target antigen may be isolated. In embodiments, tumor-infiltrating lymphocytes (TILs) or peripheral blood mononuclear cells (PBMCs) may be cultured in the presence of antigen-presenting cells (APCs) pulsed with a peptide representing an epitope known to elicit a dominant T cell response when presented in the context of a defined HLA allele. High-affinity clones may be then selected on the basis of MHC—peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) may be then generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the APCs include dendritic cells, macrophages, Langerhans cells and B cells, or T cells.

In embodiments, the binding element of the CAR may include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The embodiments of the present disclosure further relate to vectors in which a nucleic acid described herein is inserted. Vectors can be derived from retroviruses such as the lentivirus that are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for deliverying nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, “therapeutic amount”, or “effective amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, activated T cells are administered to a subject and then subsequently blood is redrawn (or have apheresis performed). T cells are collected, expanded, and reinfused into the subject. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells can be selected.

The administration of the pharmaceutical compositions described herein can be carried out in any convenient manner, including by aerosol inhalation, injection, ingestion, transfusion, implantation or transplantation. The pharmaceutical compositions described herein can be administered to a patient subcutaneously, intradermally, intratumorally, intranodally, intramedullary, intramuscularly, intravenously (i. v.), or intraperitoneally. In embodiments, the T cell compositions of the present disclosure are administered to a patient by intradermal or subcutaneous injection. In embodiments, the T cell compositions of the present disclosure are administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan®. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to an in vitro method for preparing modified cells. The method may include obtaining a sample of cells from the subject. For example, the sample may include T cells or T cell progenitors. The method may further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

As used herein, the term “gene fusion” refers to the fusion of at least a portion of a gene to at least a portion of an additional gene. The gene fusion need not include entire genes or exons of genes. In some instances, gene fusion is associated with alternations in cancer. A gene fusion product refers to a chimeric genomic DNA, a chimeric messenger RNA, a truncated protein or a chimeric protein resulting from a gene fusion. The gene fusion product may be detected by various methods described in U.S. Pat. No. 9,938,582, which is incorporated as a reference herein. A “gene fusion antigen” refers to a truncated protein or a chimeric protein that results from a gene fusion. In embodiments, an epitope of a gene fusion antigen may include a part of the gene fusion antigen or an immunogenic part of another antigen caused by the gene fusion. In embodiments, the gene fusion antigen interacts with, or is part of, cell membranes.

In embodiments, detection of mRNA and protein expression levels of a target molecules (e.g., CARs and cytokines) in human cells may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR T Cells Co-Cultured with Antigen-Positive Cells can be performed. CAR T cells can exhibit a killing effect on the corresponding antigen-positive cells, a decrease in the number of corresponding antigen-positive cells co-cultured with CAR T cells, and an increase in the release of IFN-γ, TNF-α, etc. as compared to control cells that did not express the corresponding antigen.

In embodiments, In Vivo Killer Assay can be performed. For example, mice may be transplanted with corresponding antigen tumor cells, and tumorigenic, transfusion of CAR T cells, and a decrease in mouse tumors and mouse blood IFN-γ, TNF-α, and other signals can be defected.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acid described herein.

Embodiments relate to an isolated cell comprising the isolated nucleic acid sequence described herein. Embodiments relate to a composition comprising a population of T cells comprising the CAR described herein. Embodiments relate to a CAR encoded by the isolated nucleic acid sequence described herein. Embodiments relate to a method of eliciting and/or enhancing T cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody can have twelve CDRs altogether for binding antigens.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC, for example, CD19, and a binding domain binding a solid tumor antigen. In embodiments, the bispecific CAR molecule can include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

The present disclosure describes an antibody that binds GCC (GUCY2C, Guanylate Cyclase 2C), wherein the antibody comprises a VHH domain comprising one of the amino acid sequences of SEQ ID NOs: 194 or 200-268 or comprising one or more CDRs having amino acid sequences of SEQ ID NOs: 191-193, 195-199, and 270-470. In embodiments, the antibody that binds GCC comprises three CDRs, wherein the CDRs are CDR1, CDR2, and CDR3. In embodiments, the VHH domain comprises one of SEQ ID NOs: 208, 237 and 225, and their CDRs are 294-296, 381-383 and 345-347, respectively.

The present disclosure describes a CAR that comprises an intracellular domain, a transmembrane domain, and an extracellular domain comprising the antibody. In embodiments, the extracellular domain comprises one or more of VHHs, and each of the VHHs can comprise CDR1, CDR2, and CDR3 comprising SEQ ID NO: 294, 295, and 296, respectively, comprises SEQ ID NO: 345-347, respectively, or comprises SEQ ID NO: 381-383, respectively. In embodiments, the extracellular domain comprises CDR1, CDR2, and CDR3. In embodiments, the extracellular domain comprises one of SEQ ID NOs: 208, 237 and 225. In embodiments, the extracellular domain comprises one of SEQ ID NOs: 237, 472, 225, and 475. In embodiments, the CAR comprises one of SEQ ID NOs: 471, 473, 474, and 476.

In embodiments, the extracellular domain of the CAR can comprise a scFv comprising a first VHH, a linker, and a second VHH, wherein the first VHH and second VHH are joined by the linker. In embodiments the first VHH and second VHH can be the same or different.

In embodiments, the CAR comprises an extracellular domain comprising a VHH derived binding domain or a VHH.

In embodiments, the antibody is a nanobody (single-domain antibody, sdAb) comprising or consisting essentially of a VHH (single variable domain on a heavy chain) domain. In embodiments, the antibody is conjugated to a cytotoxic agent, and the cytotoxic agent is a radioactive isotope or a toxin. In embodiments, the antibody is a bispecific antibody comprising a VHH domain, an antibody or antibody fragment, for example, scFv targeting CD3, and a linker.

In embodiments, the antibody comprises or consists essentially of a VHH domain and one or more constant domains, such as CH2 and CH3. In embodiments, the antibody is structurally similar to an alpaca antibody comprising or consisting essentially of a VHH domain, a CH2 domain, and a CH3 domains. In embodiments, the antibodies described herein comprising the VHH domain do not include the VL (variable light) and CL (constant light) domains.

The present disclosure describes a CAR comprising an antigen binding domain comprising the antibody that binds GCC, as described herein. In embodiments the antibody that binds GCC comprises a VHH domain, or a VHH one or more constant domains, such as CH2 or CH3, described herein. Embodiments describe a polynucleotide that encodes the antibody or the CAR. Embodiments describe a modified cell comprising the polynucleotide. In embodiments, the modified cell is a T cell or NK cell.

The present disclosure describes a population of modified immune cells comprising the CAR. In embodiments, the composition comprises a first population of cells comprising a first CAR binding a first antigen and a second population of cells comprising a second CAR binding a second antigen, wherein the second antigen is a tumor antigen and is different from the first antigen, and the first population and/or the second population of cells comprise one or more polynucleotides described herein.

The present disclosure describes the use of the composition comprising a first population and a second of population of cells or a method of using the composition to enhance the expansion of cells in a subject in need thereof or treating a subject having cancer, the method comprising: administering an effective amount of the composition to the subject, the subject having a form of cancer expressing a tumor antigen. In embodiments, expansion of the second population of cells in the subject is greater than the expansion of the second population of cells in a subject that is administered with the second population of cells but not the first population of cells. In embodiments, the expansion is measured based on numbers of the second population of cells or copy numbers of DNA encoding the second CAR. In embodiments, the cells are T cells, NK cells, macrophages, or dendritic cells. In embodiments, the first antigen comprises a cell surface molecule of a white blood cell (WBC), a tumor antigen, or a solid tumor antigen. In embodiments, the second antigen is different from the first antigen and the second antigen comprises a tumor antigen or a solid tumor antigen or a cell surface molecule of a WBC. In embodiments, the WBC is a granulocyte, a monocyte, or a lymphocyte. In embodiments, the WBC is a B cell. In embodiments, the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the WBC is CD19, CD20, CD22, or BCMA. In embodiments, the cell surface molecule of the WBC is CD19 or BCMA. In embodiments, the tumor antigen is a solid tumor antigen.

In embodiments, the modified cells comprise a nucleic acid sequence encoding a binding molecule and a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with lg and ITIM domains (TIGIT), leukocyte-associated immunoglobulin-like receptor 1 (LAIRI), natural killer cell receptor 2B4 (2B4), and CD160.

In embodiments, the modified cells described herein have a reduced expression of the endogenous TRAC gene. In embodiments, the modified cells include a nucleic acid sequence encoding hTERT, a nucleic acid encoding SV40LT, or a combination thereof. In embodiments, the modified cells include a nucleic acid sequence encoding hTERT and a nucleic acid encoding SV40LT. In embodiments, the expression of hTERT is regulated by an inducible expression system. In embodiments, the expression of SV40LT gene is regulated by an inducible expression system. In embodiments, the inducible expression system is rTTA-TRE, which increases or activates the expression of SV40LT gene, hTERT gene, or a combination thereof. In embodiments, the modified cells include a nucleic acid sequence encoding a suicide gene. In embodiments, the suicide gene includes an herpes simplex virus thymidine kinase (HSV-TK) suicide gene system, and/or the modified cell can be induced to undergo apoptosis.

In embodiments, the modified cells include a nucleic acid sequence encoding a cytokine. In embodiments, the modified cells include a nucleic acid sequence encoding IL-6, IFNY, IL-12, and/or IL-2.

The present disclosure describes an antibody that binds GCC, wherein the antibody comprises a VHH domain comprising complementarity determining region 1 (CDR1), CDR2, and CDR3, and wherein CDR1, CDR2, and CDR3 comprise SEQ ID NO: 294-296, respectively, CDR1, CDR2, and CDR3 comprise SEQ ID NO: 345-347, respectively, or CDR1, CDR2, and CDR3 comprise SEQ ID NO: 381-383, respectively. Embodiments describe a polynucleotide that encodes the antibody. Embodiments describe a modified cell comprising the polynucleotide. Embodiments describe a CAR comprising an extracellular domain comprising the antibody described above. In embodiments, the modified cell is a T cell or NK cell. In embodiments, the antibody comprises SEQ ID NO: 208, 225, or 237. In embodiments, the antibody is a nanobody. In embodiments, the antibody is conjugated to a cytotoxic agent, and the cytotoxic agent is a radioactive isotope or a toxin. In embodiments, the antibody is a bispecific antibody comprising the VHH domain, a linker, and an antibody targeting CD3. The antibody targeting CD3 can be a scFv antibody.

The present disclosure describes a composition comprising a population of the modified cells comprising a CAR comprising the antibody described above. In embodiments, the modified cells comprise a polynucleotide encoding a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B-and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD160. In embodiments, the modified cells have reduced expression of endogenous TRAC gene. In embodiments, the modified cells comprise a polynucleotide encoding hTERT or a nucleic acid encoding SV40LT, or a combination thereof. In embodiments, the modified cells comprise a polynucleotide encoding a cytokine. In embodiments, the modified cells include a polynucleotide encoding at least one of IL-6, IFNγ, IL-12, and IL-2.

In embodiments, the antibody comprises SEQ ID NO: 208, 225, or 237. In embodiments, the CAR comprises a transmembrane domain and an intracellular domain, and the extracellular domain comprises any of SEQ ID NO: 237, 225, and 471-476. In embodiments, the antibody comprises one or more of VHH domains of their sequences listed in Table 2.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### EXAMPLES

Various nanoantibodies targeting GCC have been generated. Methods of preparing the nanoantibodies may be found in Bever CS , Dong J X, Vasylieva N, et al. VHH antibodies: emerging reagents for the analysis of environmental chemicals, Anal Bioanal Chem. 2016;408 (22): 5985-6002, doi: 10.1007/s00216-016-9585-x; Bao, C., Gao, Q., Li, L. -L., Han, L., Zhang, B., Ding, Y., Song, Z., Zhang, R., Zhang, J., Wu, X. -H., The Application of Nanobody in CAR-T Therapy. Biomolecules 2021, 11, 238.; and Han, L., Zhang, JS., Zhou, J. et al., Single VHH-directed BCMA CAR-T cells cause remission of relapsed/refractory multiple myeloma. Leukemia (2021), all of which are incorporated herein by their entirety. VHH domains and their CDRs are identified and provided in Table 2.

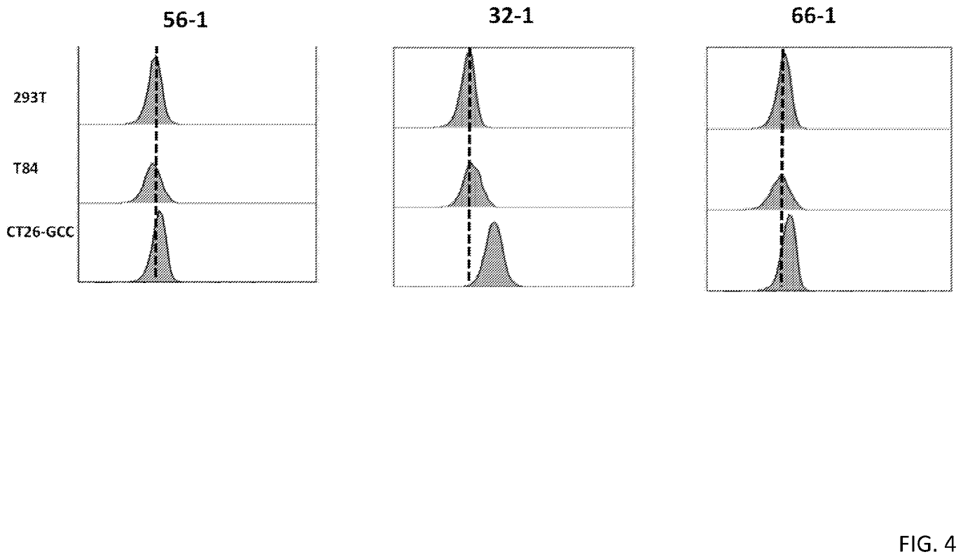

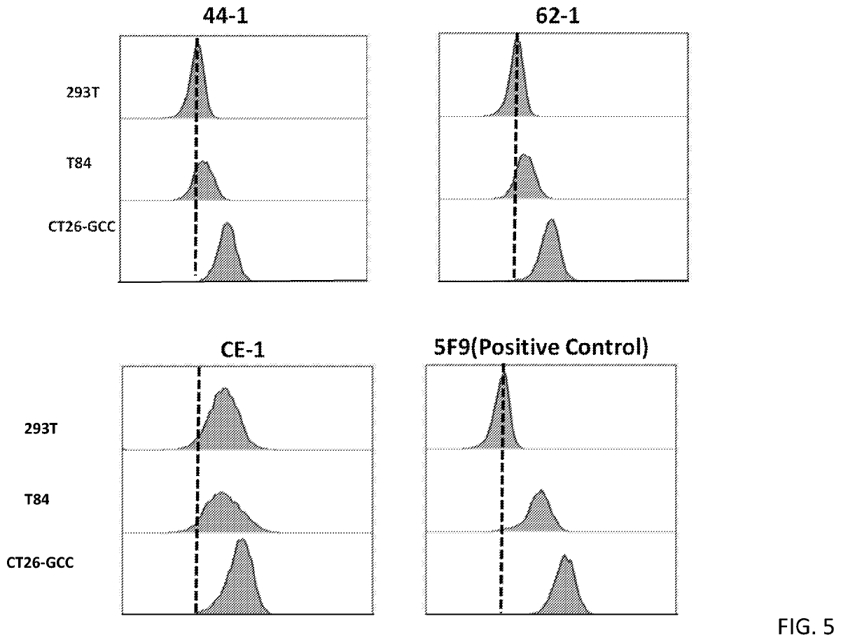

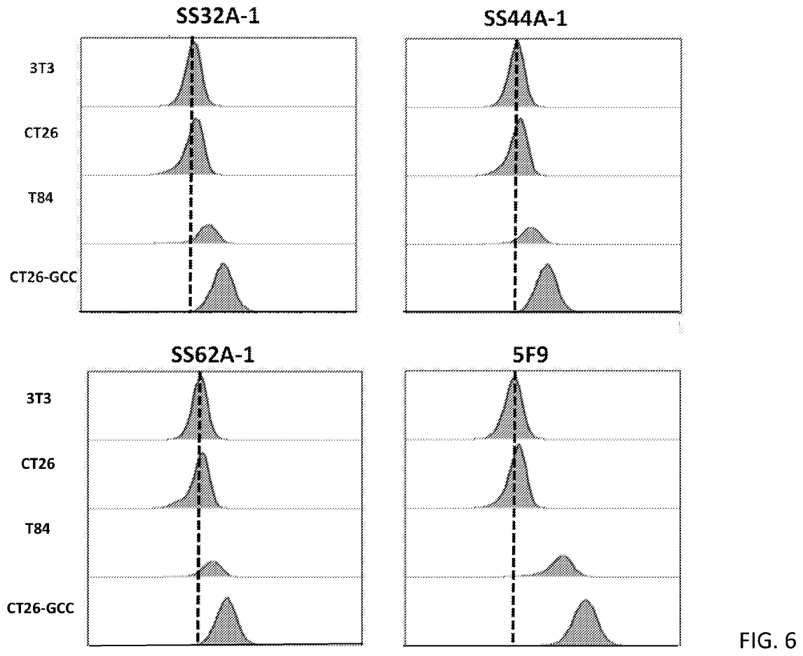

FIGS. 4 and 5 show flow cytometry results of antibody binding assay. VHH antibodies were screened using ELISA of which GCC was used as the antigen. Several VHH antibodies were identified based on the measurement of antibody affinity by the ELISA. These VHH antibodies were analyzed using GCC positive cells and GCC negative cells, and the 59E GCC antibody was used as a positive control. As shown in FIGS. 4 and 5, VHH antibodies #32 (SEQ ID NO: 237), #44 (SEQ ID NO: 225), and #62 (SEQ NO: ID: 208) specifically bind GCC positive cells but not GCC negative cells. FIG. 6 shows the flow cytometry results of the antibody binding assay. Bivalent antibodies of VHH antibodies #32, #64, #44, and #62 were generated, and these bivalent antibodies specifically bind GCC positive cells but not GCC negative cells.

VHH antibody #32, #64, #44, and #62 were selected for construction of CAR T cells. Lentiviral vectors that encode individual CAR molecules were generated and transduced into T cells, and the expression of the CARs were confirmed by flow cytometry assay. Further, these CAR T cells and GCC expressing cells were co-cultured, and the response of CAR T cells (e.g., cytokine release) induced by the GCC expressing cells were observed. Techniques related to cell cultures and construction of cytotoxic T lymphocyte assay may be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in their entirety.

TABLE 4
VHH Domains and Their CDRs

| | VHH | | VHH |
|---|---|---|---|
| SEQ ID NO | or CDR | SEQ ID NO | or CDR |
| SEQ ID NO: 200 | VHH | SEQ ID NO: 235 | VHH |
| SEQ ID NO: 270 | CDR1 | SEQ ID NO: 375 | CDR1 |
| SEQ ID NO: 271 | CDR2 | SEQ ID NO: 376 | CDR2 |
| SEQ ID NO: 272 | CDR3 | SEQ ID NO: 377 | CDR3 |
| SEQ ID NO: 201 | VHH | SEQ ID NO: 236 | VHH |
| SEQ ID NO: 273 | CDR1 | SEQ ID NO: 378 | CDR1 |
| SEQ ID NO: 274 | CDR2 | SEQ ID NO: 379 | CDR2 |
| SEQ ID NO: 275 | CDR3 | SEQ ID NO: 380 | CDR3 |
| SEQ ID NO: 202 | VHH | SEQ ID NO: 237 | VHH |
| SEQ ID NO: 276 | CDR1 | SEQ ID NO: 381 | CDR1 |
| SEQ ID NO: 277 | CDR2 | SEQ ID NO: 382 | CDR2 |
| SEQ ID NO: 278 | CDR3 | SEQ ID NO: 383 | CDR3 |
| SEQ ID NO: 203 | VHH | SEQ ID NO: 238 | VHH |
| SEQ ID NO: 279 | CDR1 | SEQ ID NO: 384 | CDR1 |
| SEQ ID NO: 280 | CDR2 | SEQ ID NO: 385 | CDR2 |
| SEQ ID NO: 281 | CDR3 | SEQ ID NO: 386 | CDR3 |
| SEQ ID NO: 204 | VHH | SEQ ID NO: 239 | VHH |
| SEQ ID NO: 282 | CDR1 | SEQ ID NO: 387 | CDR1 |
| SEQ ID NO: 283 | CDR2 | SEQ ID NO: 388 | CDR2 |
| SEQ ID NO: 284 | CDR3 | SEQ ID NO: 389 | CDR3 |
| SEQ ID NO: 205 | VHH | SEQ ID NO: 240 | VHH |
| SEQ ID NO: 285 | CDR1 | SEQ ID NO: 390 | CDR1 |
| SEQ ID NO: 286 | CDR2 | SEQ ID NO: 391 | CDR2 |
| SEQ ID NO: 287 | CDR3 | SEQ ID NO: 392 | CDR3 |
| SEQ ID NO: 206 | VHH | SEQ ID NO: 241 | VHH |
| SEQ ID NO: 288 | CDR1 | SEQ ID NO: 393 | CDR1 |
| SEQ ID NO: 289 | CDR2 | SEQ ID NO: 394 | CDR2 |
| SEQ ID NO: 290 | CDR3 | SEQ ID NO: 395 | CDR3 |
| SEQ ID NO: 207 | VHH | SEQ ID NO: 242 | VHH |
| SEQ ID NO: 291 | CDR1 | SEQ ID NO: 396 | CDR1 |
| SEQ ID NO: 292 | CDR2 | SEQ ID NO: 397 | CDR2 |
| SEQ ID NO: 293 | CDR3 | SEQ ID NO: 398 | CDR3 |
| SEQ ID NO: 208 | VHH | SEQ ID NO: 243 | VHH |
| SEQ ID NO: 294 | CDR1 | SEQ ID NO: 399 | CDR1 |
| SEQ ID NO: 295 | CDR2 | SEQ ID NO: 400 | CDR2 |
| SEQ ID NO: 296 | CDR3 | SEQ ID NO: 401 | CDR3 |
| SEQ ID NO: 209 | VHH | SEQ ID NO: 244 | VHH |
| SEQ ID NO: 297 | CDR1 | SEQ ID NO: 402 | CDR1 |
| SEQ ID NO: 298 | CDR2 | SEQ ID NO: 403 | CDR2 |
| SEQ ID NO: 299 | CDR3 | SEQ ID NO: 404 | CDR3 |
| SEQ ID NO: 210 | VHH | SEQ ID NO: 245 | VHH |
| SEQ ID NO: 300 | CDR1 | SEQ ID NO: 405 | CDR1 |
| SEQ ID NO: 301 | CDR2 | SEQ ID NO: 406 | CDR2 |
| SEQ ID NO: 302 | CDR3 | SEQ ID NO: 407 | CDR3 |
| SEQ ID NO: 211 | VHH | SEQ ID NO: 246 | VHH |
| SEQ ID NO: 303 | CDR1 | SEQ ID NO: 408 | CDR1 |
| SEQ ID NO: 304 | CDR2 | SEQ ID NO: 409 | CDR2 |
| SEQ ID NO: 305 | CDR3 | SEQ ID NO: 410 | CDR3 |
| SEQ ID NO: 212 | VHH | SEQ ID NO: 247 | VHH |
| SEQ ID NO: 306 | CDR1 | SEQ ID NO: 411 | CDR1 |
| SEQ ID NO: 307 | CDR2 | SEQ ID NO: 412 | CDR2 |
| SEQ ID NO: 308 | CDR3 | SEQ ID NO: 413 | CDR3 |
| SEQ ID NO: 213 | VHH | SEQ ID NO: 248 | VHH |
| SEQ ID NO: 309 | CDR1 | SEQ ID NO: 414 | CDR1 |
| SEQ ID NO: 310 | CDR2 | SEQ ID NO: 415 | CDR2 |
| SEQ ID NO: 311 | CDR3 | SEQ ID NO: 416 | CDR3 |
| SEQ ID NO: 214 | VHH | SEQ ID NO: 249 | VHH |
| SEQ ID NO: 312 | CDR1 | SEQ ID NO: 417 | CDR1 |
| SEQ ID NO: 313 | CDR2 | SEQ ID NO: 418 | CDR2 |
| SEQ ID NO: 314 | CDR3 | SEQ ID NO: 419 | CDR3 |
| SEQ ID NO: 215 | VHH | SEQ ID NO: 250 | VHH |
| SEQ ID NO: 315 | CDR1 | SEQ ID NO: 420 | CDR1 |
| SEQ ID NO: 316 | CDR2 | SEQ ID NO: 421 | CDR2 |
| SEQ ID NO: 317 | CDR3 | SEQ ID NO: 422 | CDR3 |
| SEQ ID NO: 216 | VHH | SEQ ID NO: 251 | VHH |
| SEQ ID NO: 318 | CDR1 | SEQ ID NO: 423 | CDR1 |
| SEQ ID NO: 319 | CDR2 | SEQ ID NO: 424 | CDR2 |
| SEQ ID NO: 320 | CDR3 | SEQ ID NO: 425 | CDR3 |
| SEQ ID NO: 217 | VHH | SEQ ID NO: 252 | VHH |
| SEQ ID NO: 321 | CDR1 | SEQ ID NO: 426 | CDR1 |
| SEQ ID NO: 322 | CDR2 | SEQ ID NO: 427 | CDR2 |
| SEQ ID NO: 323 | CDR3 | SEQ ID NO: 428 | CDR3 |
| SEQ ID NO: 218 | VHH | SEQ ID NO: 253 | VHH |
| SEQ ID NO: 324 | CDR1 | SEQ ID NO: 429 | CDR1 |
| SEQ ID NO: 325 | CDR2 | SEQ ID NO: 430 | CDR2 |
| SEQ ID NO: 326 | CDR3 | SEQ ID NO: 431 | CDR3 |
| SEQ ID NO: 219 | VHH | SEQ ID NO: 254 | VHH |
| SEQ ID NO: 327 | CDR1 | SEQ ID NO: 432 | CDR1 |
| SEQ ID NO: 328 | CDR2 | SEQ ID NO: 433 | CDR2 |
| SEQ ID NO: 329 | CDR3 | SEQ ID NO: 434 | CDR3 |
| SEQ ID NO: 220 | VHH | SEQ ID NO: 255 | VHH |
| SEQ ID NO: 330 | CDR1 | SEQ ID NO: 435 | CDR1 |
| SEQ ID NO: 331 | CDR2 | SEQ ID NO: 436 | CDR2 |
| SEQ ID NO: 332 | CDR3 | SEQ ID NO: 437 | CDR3 |
| SEQ ID NO: 221 | VHH | SEQ ID NO: 256 | VHH |
| SEQ ID NO: 333 | CDR1 | SEQ ID NO: 438 | CDR1 |
| SEQ ID NO: 334 | CDR2 | SEQ ID NO: 439 | CDR2 |
| SEQ ID NO: 335 | CDR3 | SEQ ID NO: 440 | CDR3 |
| SEQ ID NO: 222 | VHH | SEQ ID NO: 257 | VHH |
| SEQ ID NO: 336 | CDR1 | SEQ ID NO: 441 | CDR1 |
| SEQ ID NO: 337 | CDR2 | SEQ ID NO: 442 | CDR2 |
| SEQ ID NO: 338 | CDR3 | SEQ ID NO: 443 | CDR3 |
| SEQ ID NO: 223 | VHH | SEQ ID NO: 258 | VHH |
| SEQ ID NO: 339 | CDR1 | SEQ ID NO: 444 | CDR1 |
| SEQ ID NO: 340 | CDR2 | SEQ ID NO: 445 | CDR2 |
| SEQ ID NO: 341 | CDR3 | SEQ ID NO: 446 | CDR3 |
| SEQ ID NO: 224 | VHH | SEQ ID NO: 259 | VHH |
| SEQ ID NO: 342 | CDR1 | SEQ ID NO: 447 | CDR1 |
| SEQ ID NO: 343 | CDR2 | SEQ ID NO: 448 | CDR2 |
| SEQ ID NO: 344 | CDR3 | SEQ ID NO: 449 | CDR3 |
| SEQ ID NO: 225 | VHH | SEQ ID NO: 260 | VHH |
| SEQ ID NO: 345 | CDR1 | SEQ ID NO: 450 | CDR1 |
| SEQ ID NO: 346 | CDR2 | SEQ ID NO: 451 | CDR2 |
| SEQ ID NO: 347 | CDR3 | SEQ ID NO: 452 | CDR3 |
| SEQ ID NO: 226 | VHH | SEQ ID NO: 261 | VHH |
| SEQ ID NO: 348 | CDR1 | SEQ ID NO: 453 | CDR1 |
| SEQ ID NO: 349 | CDR2 | SEQ ID NO: 454 | CDR2 |
| SEQ ID NO: 350 | CDR3 | SEQ ID NO: 455 | CDR3 |
| SEQ ID NO: 227 | VHH | SEQ ID NO: 262 | VHH |
| SEQ ID NO: 351 | CDR1 | SEQ ID NO: 456 | CDR1 |
| SEQ ID NO: 352 | CDR2 | SEQ ID NO: 457 | CDR2 |
| SEQ ID NO: 353 | CDR3 | SEQ ID NO: 458 | CDR3 |
| SEQ ID NO: 228 | VHH | SEQ ID NO: 263 | VHH |
| SEQ ID NO: 354 | CDR1 | SEQ ID NO: 459 | CDR1 |
| SEQ ID NO: 355 | CDR2 | SEQ ID NO: 460 | CDR2 |
| SEQ ID NO: 356 | CDR3 | SEQ ID NO: 461 | CDR3 |
| SEQ ID NO: 229 | VHH | SEQ ID NO: 264 | VHH |
| SEQ ID NO: 357 | CDR1 | SEQ ID NO: 462 | CDR1 |
| SEQ ID NO: 358 | CDR2 | SEQ ID NO: 463 | CDR2 |
| SEQ ID NO: 359 | CDR3 | SEQ ID NO: 464 | CDR3 |
| SEQ ID NO: 230 | VHH | SEQ ID NO: 265 | VHH |
| SEQ ID NO: 360 | CDR1 | SEQ ID NO: 465 | CDR1 |
| SEQ ID NO: 361 | CDR2 | SEQ ID NO: 466 | CDR2 |
| SEQ ID NO: 362 | CDR3 | SEQ ID NO: 467 | CDR3 |
| SEQ ID NO: 231 | VHH | SEQ ID NO: 266 | VHH |
| SEQ ID NO: 363 | CDR1 | SEQ ID NO: 468 | CDR1 |
| SEQ ID NO: 364 | CDR2 | SEQ ID NO: 469 | CDR2 |
| SEQ ID NO: 365 | CDR3 | SEQ ID NO: 470 | CDR3 |
| SEQ ID NO: 232 | VHH | SEQ ID NO: 267 | VHH |
| SEQ ID NO: 366 | CDR1 | SEQ ID NO: 471 | CDR1 |
| SEQ ID NO: 367 | CDR2 | SEQ ID NO: 199 | CDR2 |
| SEQ ID NO: 368 | CDR3 | SEQ ID NO: 198 | CDR3 |
| SEQ ID NO: 233 | VHH | SEQ ID NO: 268 | VHH |
| SEQ ID NO: 369 | CDR1 | SEQ ID NO: 197 | CDR1 |
| SEQ ID NO: 370 | CDR2 | SEQ ID NO: 196 | CDR2 |
| SEQ ID NO: 371 | CDR3 | SEQ ID NO: 195 | CDR3 |
| SEQ ID NO: 234 | VHH | SEQ ID NO: 194 | VHH |
| SEQ ID NO: 372 | CDR1 | SEQ ID NO: 193 | CDR1 |
| SEQ ID NO: 373 | CDR2 | SEQ ID NO: 192 | CDR2 |
| SEQ ID NO: 374 | CDR3 | SEQ ID NO: 191 | CDR3 |

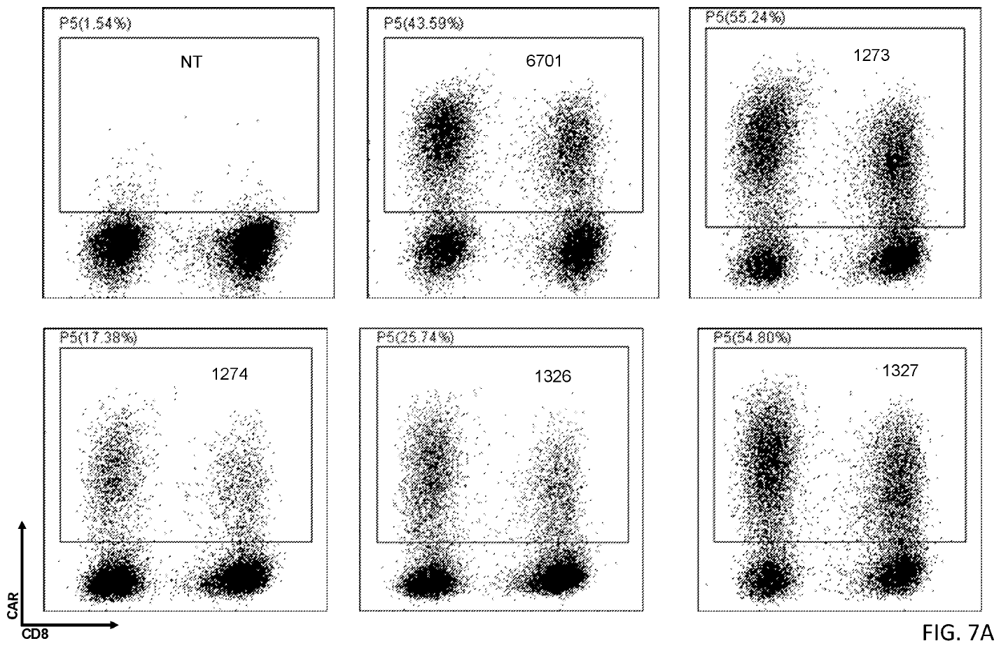

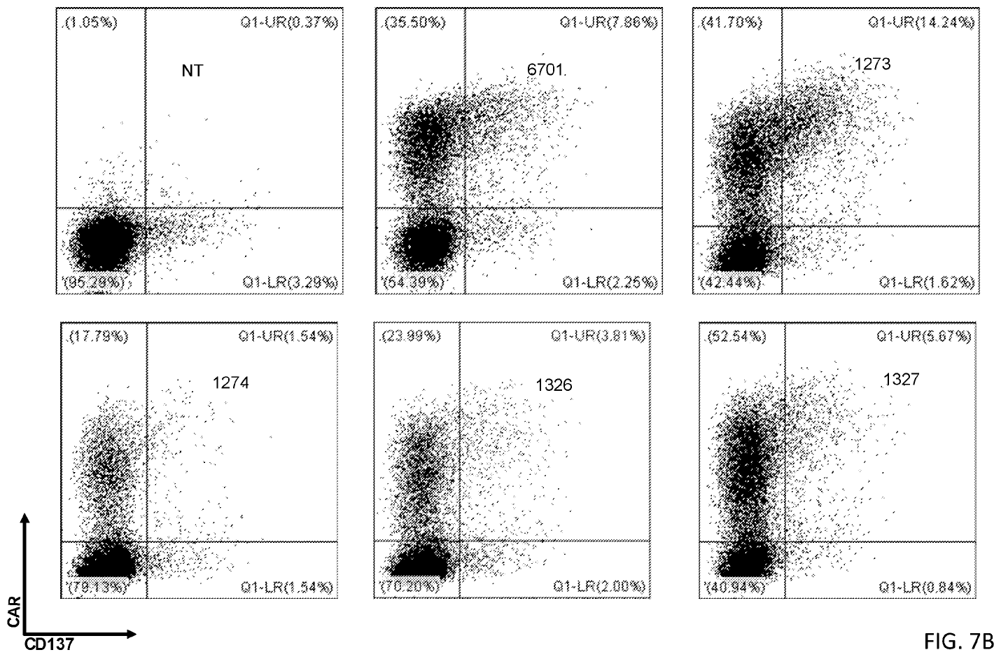

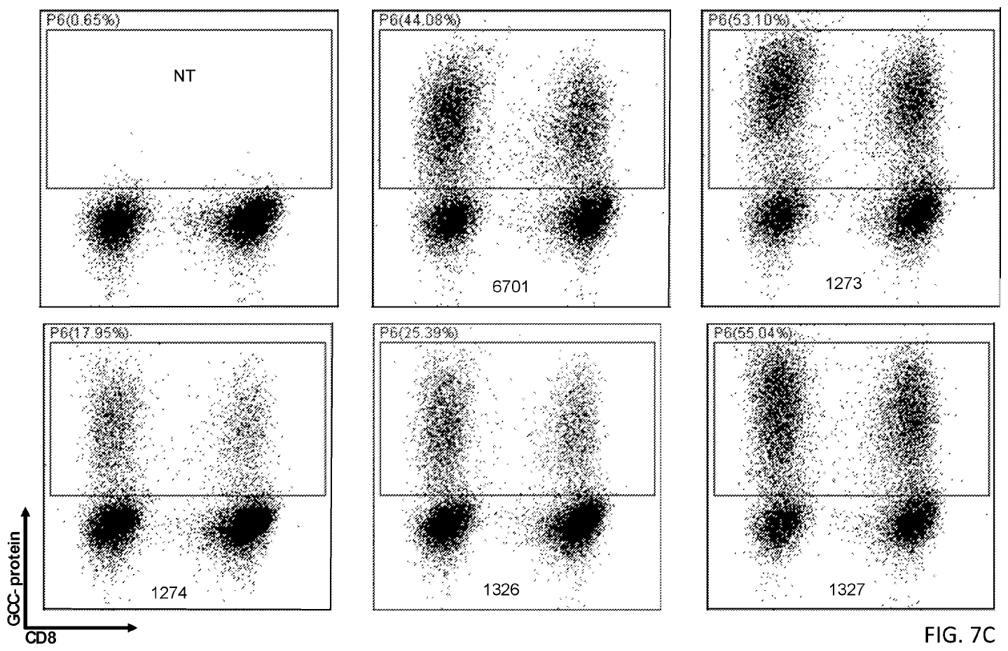

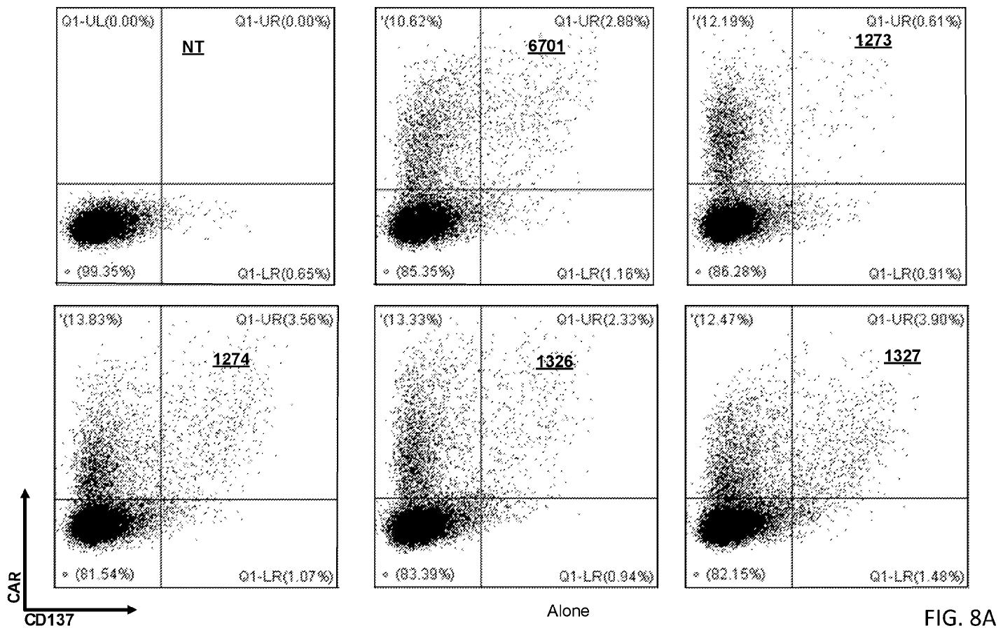

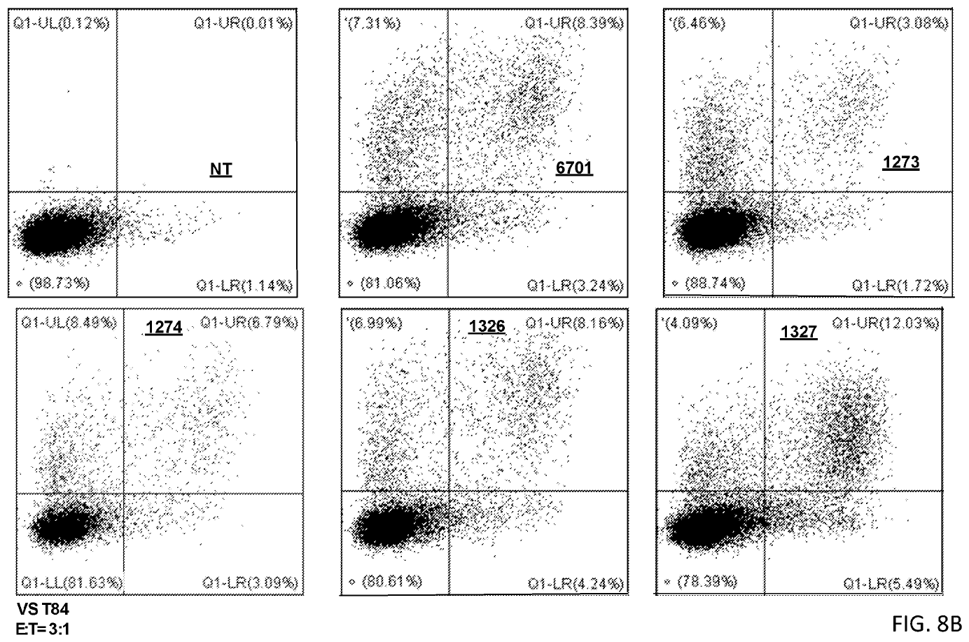

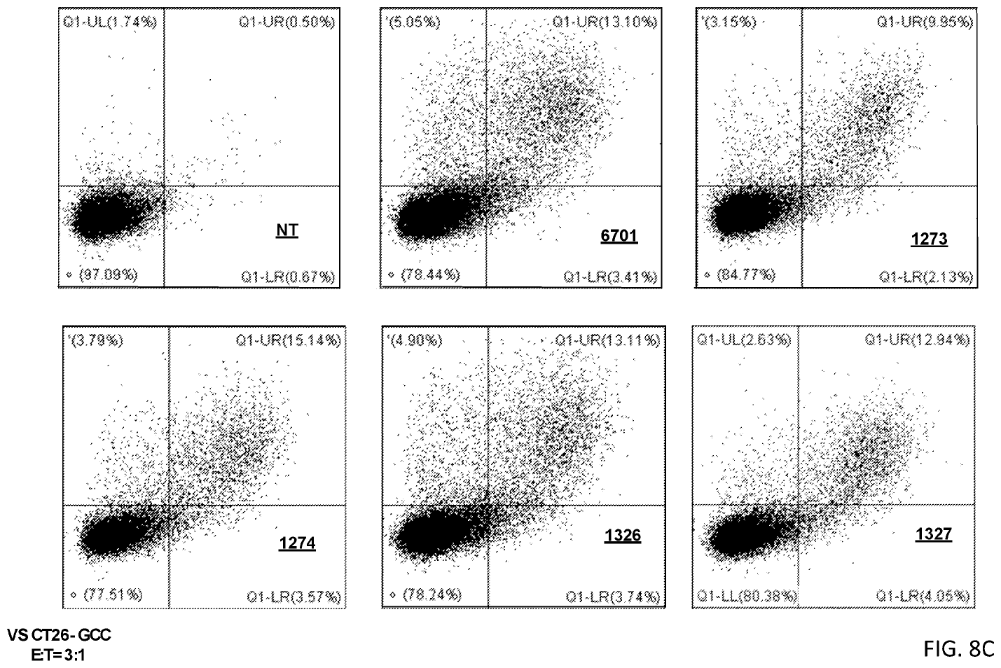

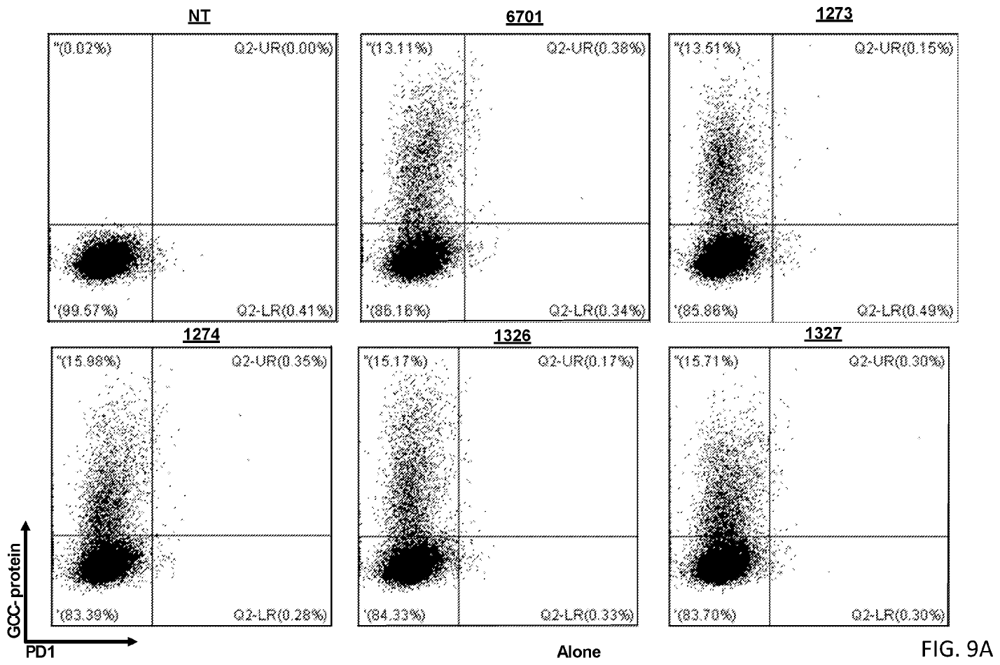

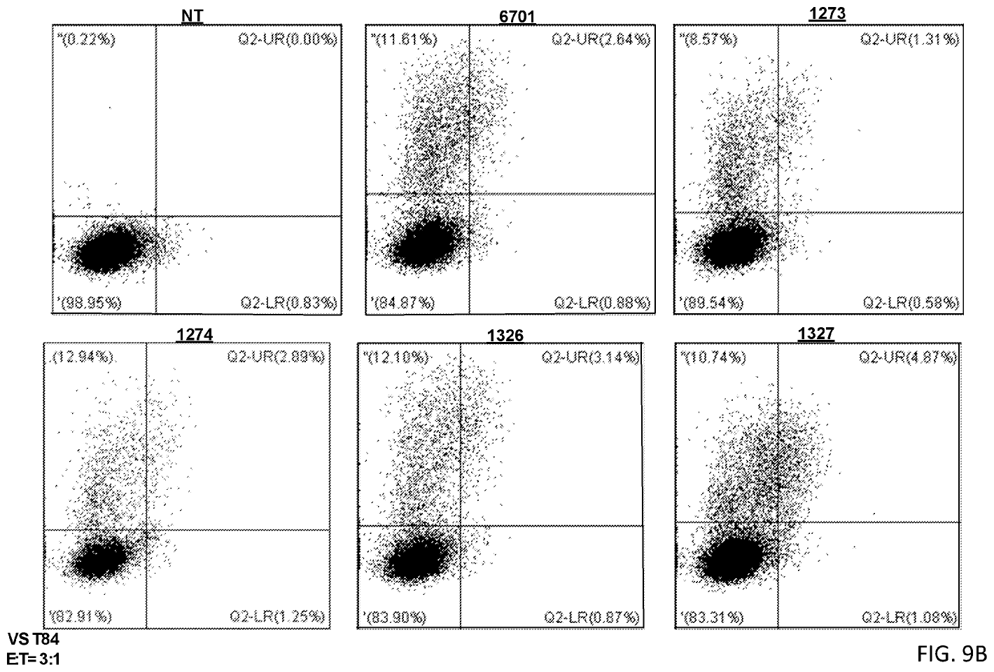

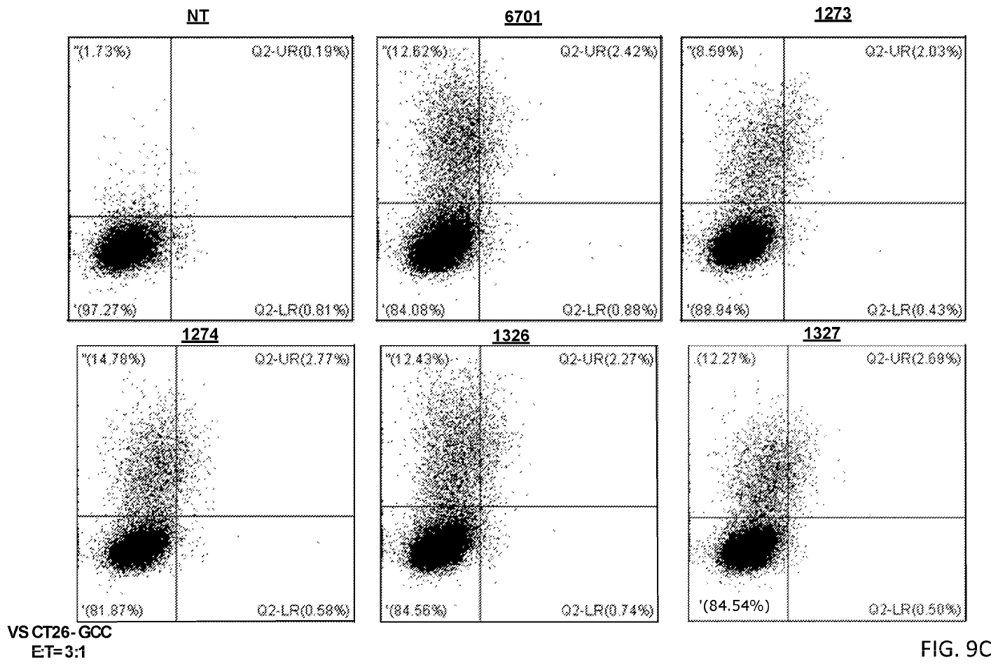

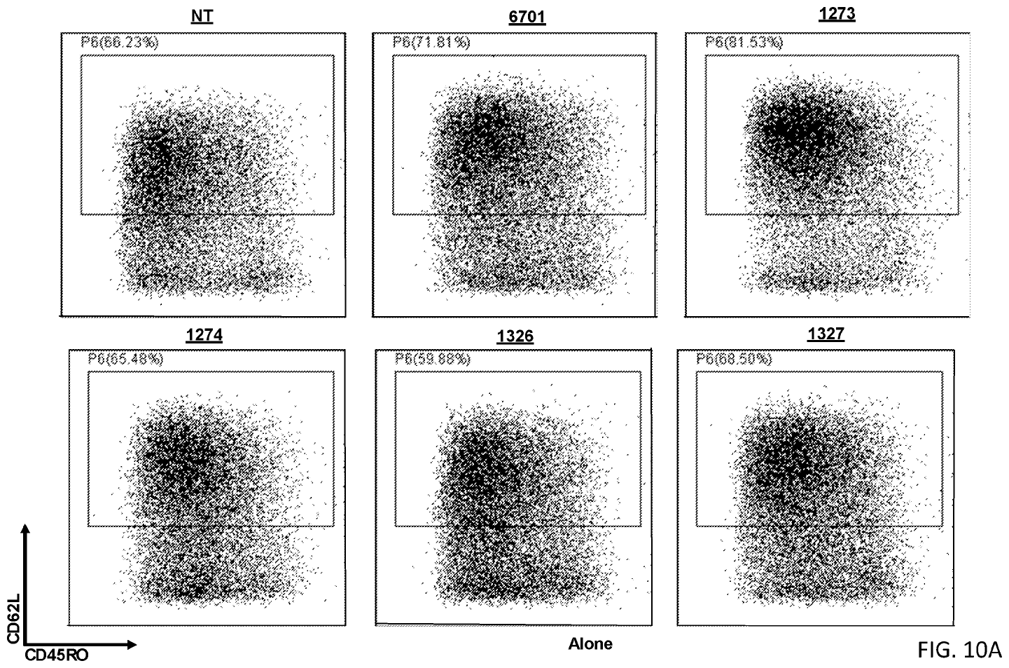

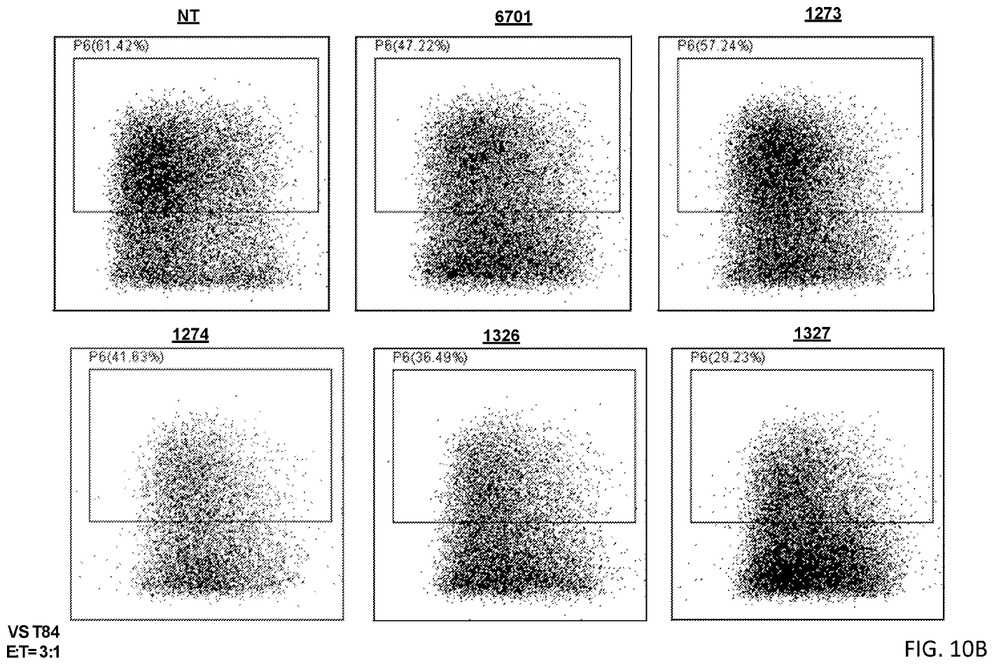

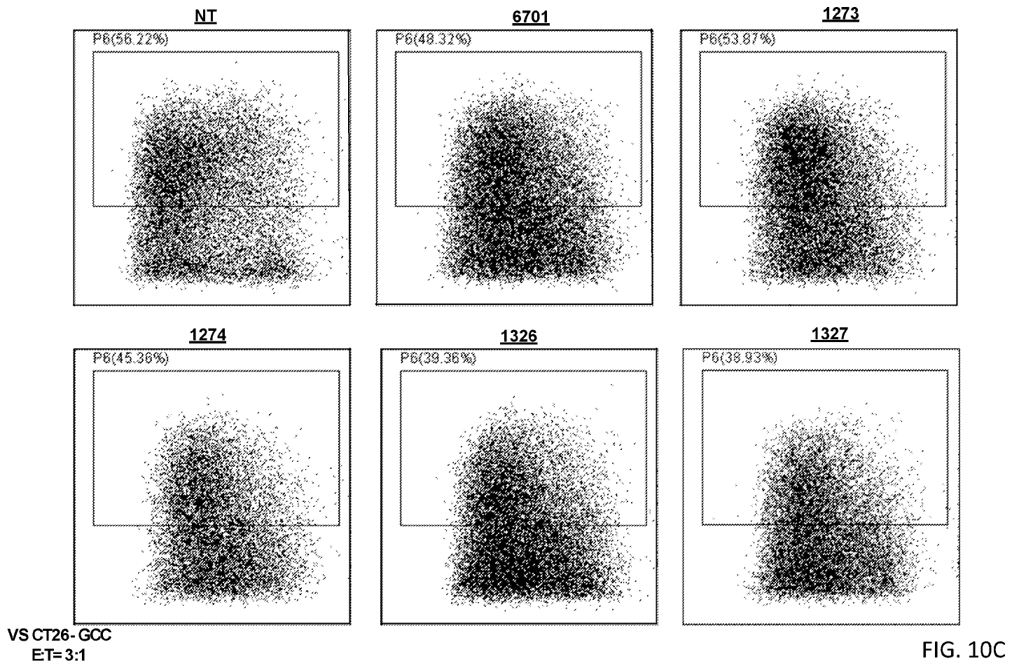

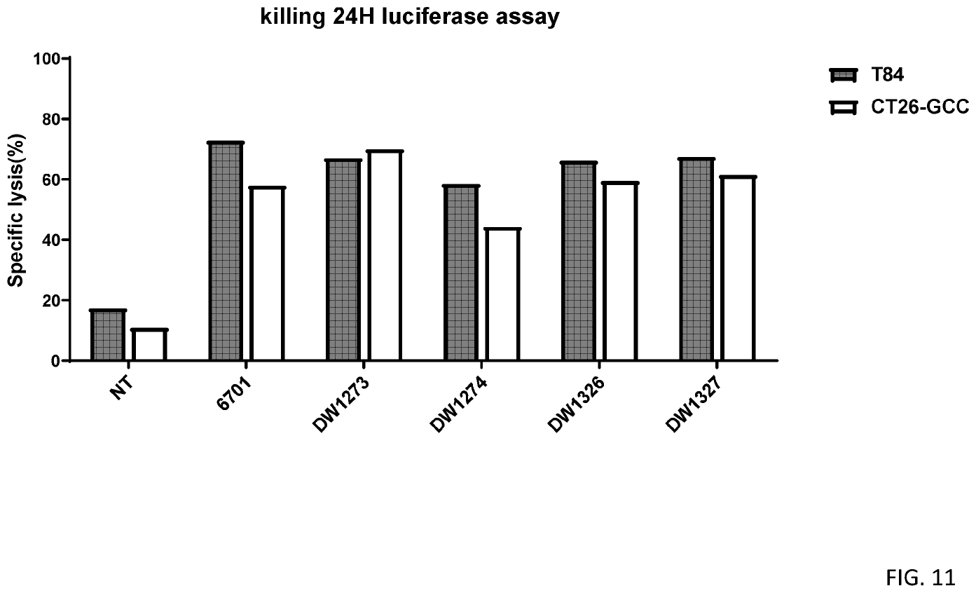

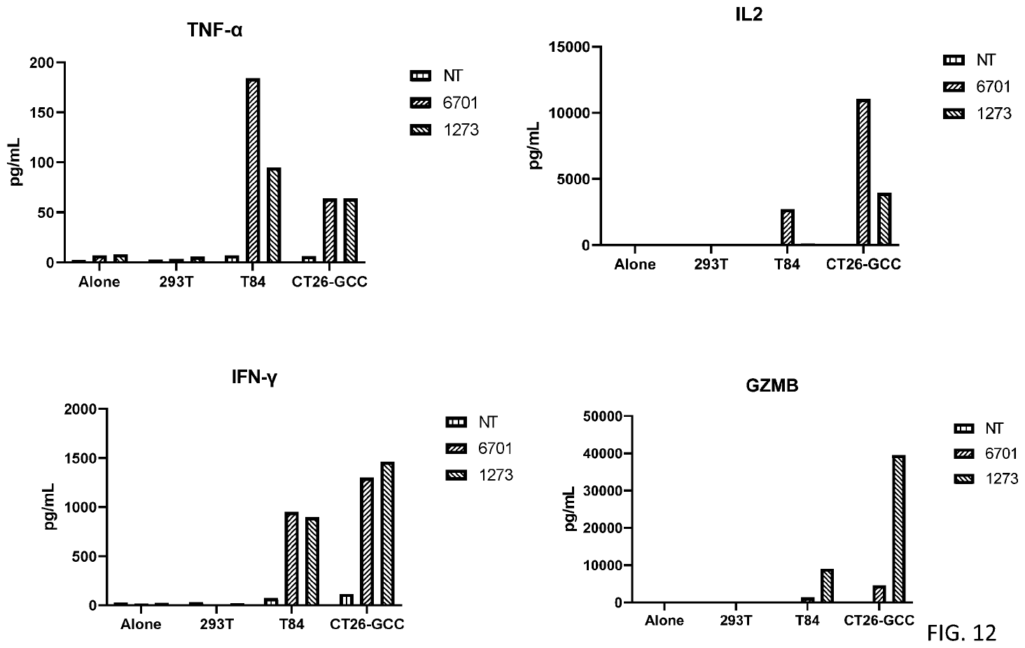

T84 and T26-GCC are cell lines overexpressing GCC, and 293T is a cell line that does not express GCC. After co-culturing with GCC-positive cells, 6701 and 1273 released cytokines, including IL2, TNF-α, IFN-g, and GZMB. FIGS. 7A, 7B, and 7C show GCC CAR expression and activation 7 days after the transduction. FIGS. 8A, 8B, and 8C show activation of GCC CAR T cells after co-culturing with GCC-positive cells. FIGS. 9A, 9B, and 9C show exhaustion of GCC CAR T cells after co-culturing with GCC-positive cells for 24 hours. FIGS. 10A, 10B, and 10C show cells with memory-like phenotypes of GCC CAR T cells after co-culturing with GCC-positive cells. The phenotypes were measured using a depletion marker: CD62L and 1273 showed a stronger memory phenotype than other cells. FIG. 11 shows a killing assay of GCC CAR T cells after co-culturing with GCC-positive cells. FIG. 12 shows the release of cytokines after GCC CAR co-cultivation. Table 3 shows information of CAR T cells, vectors encoding CARs, and their sequences.

TABLE 3

| Cells References | Type | SEQ ID NO | Antibodies |
|---|---|---|---|
| 1273 | scfv | 237 | #32 |
| 1274 | CAR | 471 | #32 |
| 1274 | scfv | 472 | #32 |
| 1274 | CAR | 473 | #32 |
| 1326 | scfv | 225 | #44 |
| 1326 | CAR | 474 | #44 |
| 1327 | scfv | 475 | #44 |
| 1327 | CAR | 476 | #44 |
| 6701 | scfv | 477 | 5F9 |
| 6701 | CAR | 478 | 5F9 |

Related sequences, compositions, and methods of treating cancer are provided in this Application and Innovative Cellular Therapeutics' PCT Patent Publication NOS: WO2016138846, WO2018126369, WO2017167217, WO2019140100, WO2020146743, WO2021216731, WO2020106843, WO2020047306, and WO2022150831 and US Patent Publication NOS: US20210060069 and US20210100841, which are incorporated by reference in their entirety.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. An isolated polynucleotide encoding a chimeric antigen receptor (CAR) that binds to GCC, wherein the CAR comprises: an extracellular domain that binds to GCC; a transmembrane domain; and an intracellular domain, the extracellular domain comprising SEQ ID NO: 237, 472, 225, or 475.

2. The isolated polynucleotide of claim 1, wherein the polynucleotide comprises SEQ ID NO: 471, 473, 474, or 476.

3. The isolated polynucleotide of claim 1, wherein the intracellular domain comprises a signaling domain comprising CD3-zeta domain.

4. The isolated polynucleotide of claim 1, wherein the intracellular domain further comprises a 4-1BB or CD28 co-stimulatory domain.

5. A vector comprising the isolated polynucleotide of claim 1.

6. The vector of claim 5, wherein the vector is a viral vector.

7. The vector of claim 6, wherein the viral vector is a lentiviral, retroviral, or adeno-associated viral vector.

8. A composition comprising a population of genetically modified T or NK cells comprising the polynucleotide of claim 1.

9. The composition of claim 8, wherein the population of genetically modified T or NK cells induces release of one or more cytokines when co-cultured with cells expressing GCC.

10. The composition of claim 9, wherein the one or more cytokines comprise IFN-γ, TNF-α, IL-2, and GZMB.

11. A method of inducing release of one or more cytokines from cells expressing GCC, comprising:
contacting a population of T cells comprising the polynucleotide of of claim 1, to obtain modified T cells expressing a CAR that binds GCC; and
co-culturing the modified T cells with cells expressing GCC, thereby inducing the release of one or more cytokines from the cells expressing GCC.

12. The method of claim 11, wherein the cells expressing GCC are cancer cells.

13. The method of claim 11, wherein the one or more cytokines comprise IFN-γ, TNF-α, IL-2, or GZMB.

14. A method of treating cancer in a subject, comprising:
obtaining T cells from the subject;
contacting a population of T cells comprising the polynucleotide of claim 1, to obtain modified T cells expressing a CAR that binds GCC; and
administering an effective amount of the modified T cells to the subject, wherein the cancer comprises cells expressing GCC.

15. The method of claim 14, further comprising expanding the modified T cells ex vivo prior to administration.

16. The method of claim 14, wherein the cancer is colorectal, gastric, esophageal, bladder, pancreatic, cervical, ovarian, breast, lung, or prostate cancer.

17. The method of claim 14, further comprising administering an immunosuppressive agent, chemotherapy, or radiation to the subject.


SEQUENCE LISTING (478 sequences)

SEQ ID NO: 1 - protein, 184 aa, Homo sapiens
MAPLLPIRTLPLILILLALLSPGAADFNISSLSGLLSPALTESLLVALPPCHLTGGNATLMVRRANDSKVVTSSFVVPPCRGRRELVSVVDSGAGFTVTRLSAYQVTNLVPGTKFYISYLVKKGTATESSREIPMSTLPRRNMESIGLGMARTGGMVVITVLLSVAMFLLVLGFIIALALGSRK

SEQ ID NO: 2 - protein, 909 aa, Homo sapiens
MAARPLPVSPARALLLALAGALLAPCEARGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 3 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 4 - protein, 418 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQVLKVIFAVAFCLISAVLMVLLFIHIRRGLCWQRESYGNI

SEQ ID NO: 5 - protein, 560 aa, Homo sapiens
MKMQKGNVLLMFGLLLHLEAATNSNETSTSANTGSSVISSGASTATNSGSSVTSSGVSTATISGSSVTSNGVSIVTNSEFHTTSSGISTATNSEFSTVSSGISIATNSESSTTSSGASTATNSESSTPSSGASTATNSDSSTTSSGASTATNSDSSTTSSEASTATNSESSTTSSGASTATNSESSTVSSRASTATNSESSTTSSGASTATNSESRTTSNGAGTATNSESSTTSSGASTATNSESSTPSSGAGTATNSESSTTSSGAGTATNSESSTVSSGISTVTNSESSTPSSGANTATNSESSTTSSGANTATNSDSSTTSSGASTATNSESSTTSSGASTATNSESSTTSSGASTATNSGSSTTSSGTSTATNSESSTVSSGASTATTSESSTTSSGASTATNSESSTVSSGASTATNSESSTTSSGANTATNSGSSVTSAGSGTAALTGMHTTSHSASTAVSEAKPGGSLVPWEIFLITLVSVVAAVGLFAGLFFCVRNSLSLRNTFNTAVYHPHGLNHGLGPGPGGNHGAPHRPRWSPNWFWRRPVSSIAMEMS

SEQ ID NO: 6 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 7 - protein, 267 aa, Homo sapiens
MMSSKPTSHAEVNETIPNPYPPSSFMAPGFQQPLGSINLENQAQGAQRAQPYGITSPGIFASSQPGQGNIQMINPSVGTAVMNFKEEAKALGVIQIMVGLMHIGFGIVLCLISFSFREVLGFASTAVIGGYPFWGGLSFIISGSLSVSASKELSRCLVKGSLGMNIVSSILAFIGVILLLVDMCINGVAGQDYWAVLSGKGISATLMIFSLLEFFVACATAHFANQANTTTNMSVLVIPNMYESNPVTPASSSAPPRCNNYSANAPK

SEQ ID NO: 8 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 9 - protein, 520 aa, Homo sapiens
MEFHNGGHVSGIGGFLVSLTSRMKPHTLAVTPALIFAITVATIGSFQFGYNTGVINAPETIIKEFINKTLTDKANAPPSEVLLTNLWSLSVAIFSVGGMIGSFSVGLFVNRFGRRNSMLIVNLLAATGGCLMGLCKIAESVEMLILGRLVIGLFCGLCTGFVPMYIGEISPTALRGAFGTLNQLGIVIGILVAQIFGLELILGSEELWPVLLGFTILPAILQSAALPCCPESPRFLLINRKKEENATRILQRLWGTQDVSQDIQEMKDESARMSQEKQVTVLELFRVSSYRQPIIISIVLQLSQQLSGINAVFYYSTGIFKDAGVQQPIYATISAGVVNTIFTLLSLFLVERAGRRTLHMIGLGGMAFCSTLMTVSLLLKNHYNGMSFVCIGAILVFVACFEIGPGPIPWFIVAELFSQGPRPAAMAVAGCSNWTSNFLVGLLFPSAAYYLGAYVFIIFTGFLITFLAFTFFKVPETRGRTFEDITRAFEGQAHGADRSGKDGVMGMNSIEPAKETTTNV

SEQ ID NO: 10 - protein, 221 aa, Homo sapiens
MLTLEDKDMKGFSWAIVPALTSLGYLIILVVSIFPFWVRLTNEESHEVFFSGLFENCFNAKCWKPRPLSIYIILGRVFLLSAVFLAFVTTFIMMPFASEFFPRTWKQNFVLACISFFTGACAFLALVLHALEIKALRMKLGPLQFSVLWPYYVLGFGIFLFIVAGTICLIQEMVCPCWHLLSTSQSMEEDHGSLYLDNLESLGGEPSSVQKETQVTAETVI

SEQ ID NO: 11 - protein, 538 aa, Homo sapiens
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 12 - protein, 1017 aa, Homo sapiens
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 13 - protein, 775 aa, Homo sapiens
MEPPYSLTAHYDEFQEVKYVSRCGAGGARGASLPPGFPLGAARSATGARSGLPRWNRREVCLLSGLVFAAGLCAILAAMLALKYLGPVAAGGGACPEGCPERKAFARAARFLAANLDASIDPCQDFYSFACGGWLRRHAIPDDKLTYGTIAAIGEQNEERLRRLLARPGGGPGGAAQRKVRAFFRSCLDMREIERLGPRPMLEVIEDCGGWDLGGAEERPGVAARWDLNRLLYKAQGVYSAAALFSLTVSLDDRNSSRYVIRIDQDGLTLPERTLYLAQDEDSEKILAAYRVFMERVLSLLGADAVEQKAQEILQVEQQLANITVSEHDDLRRDVSSMYNKVTLGQLQKITPHLRWKWLLDQIFQEDFSEEEEVVLLATDYMQQVSQLIRSTPHRVLHNYLVWRVVVVLSEHLSPPFREALHELAQEMEGSDKPQELARVCLGQANRHFGMALGALFVHEHFSAASKAKVQQLVEDIKYILGQRLEELDWMDAETRAAARAKLQYMMVMVGYPDFLLKPDAVDKEYEFEVHEKTYFKNILNSIRFSIQLSVKKIRQEVDKSTWLLPPQALNAYYLPNKNQMVFPAGILQPTLYDPDFPQSLNYGGIGTIIGHELTHGYDDWGGQYDRSGNLLHWWTEASYSRFLRKAECIVRLYDNFTVYNQRVNGKHTLGENIADMGGLKLAYHAYQKWVREHGPEHPLPRLKYTHDQLFFIAFAQNWCIKRRSQSIYLQVLTDKHAPEHYRVLGSVSQFEEFGRAFHCPKDSPMNPAHKCSVW

SEQ ID NO: 14 - protein, 529 aa, Homo sapiens
MGPSCPVFLSFTKLSLWWLLLTPAGGEEAKRPPPRAPGDPLSSPSPTALPQGGSHTETEDRLFKHLFRGYNRWARPVPNTSDVVIVRFGLSIAQLIDVDEKNQMMTTNVWLKQEWSDYKLRWNPTDFGNITSLRVPSEMIWIPDIVLYNNADGEFAVTHMTKAHLFSTGTVHWVPPAIYKSSCSIDVTFFPFDQQNCKMKFGSWTYDKAKIDLEQMEQTVDLKDYWESGEWAIVNATGTYNSKKYDCCAEIYPDVTYAFVIRRLPLFYTINLIIPCLLISCLTVLVFYLPSDCGEKITLCISVLLSLTVFLLLITEIIPSTSLVIPLIGEYLLFTMIFVTLSIVITVFVLNVHHRSPSTHTMPHWVRGALLGCVPRWLLMNRPPPPVELCHPLRLKLSPSYHWLESNVDAEEREVVVEEEDRWACAGHVAPSVGTLCSHGHLHSGASGPKAEALLQEGELLLSPHMQKALEGVHYIADHLRSEDADSSVKEDWKYVAMVIDRIFLWLFIIVCFLGTIGLFLPPFLAGMI

SEQ ID NO: 15 - protein, 537 aa, Homo sapiens
MPHLMERMVGSGLLWLALVSCILTQASAVQRGYGNPIEASSYGLDLDCGAPGTPEAHVCFDPCQNYTLLDEPFRSTENSAGSQGCDKNMSGWYRFVGEGGVRMSETCVQVHRCQTDAPMWLNGTHPALGDGITNHTACAHWSGNCCFWKTEVLVKACPGGYHVYRLEGTPWCNLRYCTVPRDPSTVEDKCEKACRPEEECLALNSTWGCFCRQDLNSSDVHSLQPQLDCGPREIKVKVDKCLLGGLGLGEEVIAYLRDPNCSSILQTEERNWVSVTSPVQASACRNILERNQTHAIYKNTLSLVNDFIIRDTILNINFQCAYPLDMKVSLQAALQPIVSSLNVSVDGNGEFIVRMALFQDQNYTNPYEGDAVELSVESVLYVGAILEQGDTSRFNLVLRNCYATPTEDKADLVKYFIIRNSCSNQRDSTIHVEENGQSSESRFSVQMFMFAGHYDLVFLHCEIHLCDSLNEQCQPSCSRSQVRSEVPAIDLARVLDLGPITRRGAQSPGVMNGTPSTAGFLVAWPMVLLTVLLAWLF

SEQ ID NO: 16 - protein, 426 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIRDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 17 - protein, 328 aa, Homo sapiens
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 18 - protein, 620 aa, Homo sapiens
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 19 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELLRLGSHPAPARAQKPGSSGLAARGLCVLGEDNAPL

SEQ ID NO: 20 - protein, 431 aa, Homo sapiens
MQALNITPEQFSRLLRDHNLTREQFIALYRLRPLVYTPELPGRAKLALVLTGVLIFALALFGNALVFYVVTRSKAMRTVTNIFICSLALSDLLITFFCIPVTMLQNISDNWLGGAFICKMVPFVQSTAVVTEILTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAVIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 21 - protein, 335 aa, Homo sapiens
MESSFSFGVILAVLASLIIATNTLVAVAVLLLIHKNDGVSLCFTLNLAVADTLIGVAISGLLTDQLSSPSRPTQKTLCSLRMAFVTSSAAASVLTVMLITFDRYLAIKQPFRYLKIMSGFVAGACIAGLWLVSYLIGFLPLGIPMFQQTAYKGQCSFFAVFHPHFVLTLSCVGFFPAMLLFVFFYCDMLKIASMHSQQIRKMEHAGAMAGGYRSPRTPSDFKALRTVSVLIGSFALSWTPFLITGIVQVACQECHLYLVLERYLWLLGVGNSLLNPLIYAYWQKEVRLQLYHMALGVKKVLTSFLLFLSARNCGPERPRESSCHIVTISSSEFDG

SEQ ID NO: 22 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 23 - protein, 21 aa, synthetic construct
MALPVTALLLPLALLLHAARP

SEQ ID NO: 24 - protein, 15 aa, synthetic construct
GGGGSGGGGSGGGGS

SEQ ID NO: 25 - protein, 45 aa, synthetic construct
TTTPAPRPPTPAPTIASQPLSLRPEACRPAAGGAVHTRGLDFACD

SEQ ID NO: 26 - protein, 23 aa, synthetic construct
YIWAPLAGTCGVLLLSLVITLYC

SEQ ID NO: 27 - protein, 42 aa, synthetic construct
KRGRKKLLYIFKQPFMRPVQTTQEEDGCSCRFPEEEEGGCEL

SEQ ID NO: 28 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 485 aa, synthetic construct
MALPVTALLLPLALLLHAARPQIVLTQSPAIMSASLGERVTMTCTASSVVISTYLHWYQQKPGSSPKLWIYSTSNLASGVPVRFSGSGSGTSYSLTISSMEAEDAATYYCHQYHRSPWTFGGGTKLEIKGGGGSGGGGSGGGGSEVQLQQSGPELVKPGGSMKISCKASGYSFTGYTMNWVKQSHGKNLEWIGLINPYNGGISYNQKFKDKATLTMDKSSSTAYMELLSLTSEDSAIYFCARDYRYEGFDYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 241 aa, synthetic construct
QIVLTQSPAIMSASLGERVTMTCTASSVVISTYLHWYQQKPGSSPKLWIYSTSNLASGVPVRFSGSGSGTSYSLTISSMEAEDAATYYCHQYHRSPWTFGGGTKLEIKGGGGSGGGGSGGGGSEVQLQQSGPELVKPGGSMKISCKASGYSFTGYTMNWVKQSHGKNLEWIGLINPYNGGISYNQKFKDKATLTMDKSSSTAYMELLSLTSEDSAIYFCARDYRYEGFDYWGQGTLVTVSA

SEQ ID NO: 31 - protein, 108 aa, synthetic construct
QIVLTQSPAIMSASLGERVTMTCTASSVVISTYLHWYQQKPGSSPKLWIYSTSNLASGVPVRFSGSGSGTSYSLTISSMEAEDAATYYCHQYHRSPWTFGGGTKLEIK

SEQ ID NO: 32 - protein, 118 aa, synthetic construct
EVQLQQSGPELVKPGGSMKISCKASGYSFTGYTMNWVKQSHGKNLEWIGLINPYNGGISYNQKFKDKATLTMDKSSSTAYMELLSLTSEDSAIYFCARDYRYEGFDYWGQGTLVTVSA

SEQ ID NO: 33 - protein, 488 aa, synthetic construct
MALPVTALLLPLALLLHAARPENVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELKGGGGSGGGGSGGGGSEVQLQQSGPELVKPGASVKISCKASGYSFTGYFMNWVKQSHGKSLEWIGRINPYNGDTFYNQKFKGKATLTVDKSSNTAHMELRSLTSEDSAVYYCARVLFLDFDDPYLMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 244 aa, synthetic construct
ENVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELKGGGGSGGGGSGGGGSEVQLQQSGPELVKPGASVKISCKASGYSFTGYFMNWVKQSHGKSLEWIGRINPYNGDTFYNQKFKGKATLTVDKSSNTAHMELRSLTSEDSAVYYCARVLFLDFDDPYLMDYWGQGTSVTVSS

SEQ ID NO: 35 - protein, 106 aa, synthetic construct
NVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELKE

SEQ ID NO: 36 - protein, 123 aa, synthetic construct
EVQLQQSGPELVKPGASVKISCKASGYSFTGYFMNWVKQSHGKSLEWIGRINPYNGDTFYNQKFKGKATLTVDKSSNTAHMELRSLTSEDSAVYYCARVLFLDFDDPYLMDYWGQGTSVTVSS

SEQ ID NO: 37 - protein, 485 aa, synthetic construct
MALPVTALLLPLALLLHAARPDIQMTQSPASLSASVGETVTITCRASENIDSYLAWYQQKQGKSPQLLVYASTLLVDGVPSRFSGSRSGTQFSLKINSLQSEDVARYYCQHYYSIPYTFGSGTKLEIKGGGGSGGGGSGGGGSEVKLVESGGGLVKPGGSLKLSCAASGFTFNSYTMSWVRQTPAKRLEWVVTISSGGGRTYYPDSVKGRFTISRDNARNTLYLQMSSLRSEDTAMYYCIRGDYRYDGFAYWGQGTLVTVSTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 241 aa, synthetic construct
DIQMTQSPASLSASVGETVTITCRASENIDSYLAWYQQKQGKSPQLLVYASTLLVDGVPSRFSGSRSGTQFSLKINSLQSEDVARYYCQHYYSIPYTFGSGTKLEIKGGGGSGGGGSGGGGSEVKLVESGGGLVKPGGSLKLSCAASGFTFNSYTMSWVRQTPAKRLEWVVTISSGGGRTYYPDSVKGRFTISRDNARNTLYLQMSSLRSEDTAMYYCIRGDYRYDGFAYWGQGTLVTVST

SEQ ID NO: 39 - protein, 107 aa, synthetic construct
DIQMTQSPASLSASVGETVTITCRASENIDSYLAWYQQKQGKSPQLLVYASTLLVDGVPSRFSGSRSGTQFSLKINSLQSEDVARYYCQHYYSIPYTFGSGTKLEIK

SEQ ID NO: 40 - protein, 119 aa, synthetic construct
EVKLVESGGGLVKPGGSLKLSCAASGFTFNSYTMSWVRQTPAKRLEWVVTISSGGGRTYYPDSVKGRFTISRDNARNTLYLQMSSLRSEDTAMYYCIRGDYRYDGFAYWGQGTLVTVST

SEQ ID NO: 41 - protein, 488 aa, synthetic construct
MALPVTALLLPLALLLHAARPENVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELKGGGGSGGGGSGGGGSEVQLQQSGPELVKPGASVKISCKASGYSFTGYFMNWVKQSHGKSLEWIGRINPYNGDTFYNQKFKGKATLTVDKSSSTAHMELRSLTSEDSAVYYCARVLFLDFDDPYLMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 244 aa, synthetic construct
ENVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELKGGGGSGGGGSGGGGSEVQLQQSGPELVKPGASVKISCKASGYSFTGYFMNWVKQSHGKSLEWIGRINPYNGDTFYNQKFKGKATLTVDKSSSTAHMELRSLTSEDSAVYYCARVLFLDFDDPYLMDYWGQGTSVTVSS

SEQ ID NO: 43 - protein, 106 aa, synthetic construct
ENVLTQSPAIMSATLGEKVTMSCRATSNVKYMYWYQQKSGASPKLWIYYTSNLASGVPARFSGSGSGTSYSLTISSVEAADAATYYCQQFTSSPSTFGAGTKLELK

SEQ ID NO: 44 - protein, 123 aa, synthetic construct
EVQLQQSGPELVKPGASVKISCKASGYSFTGYFMNWVKQSHGKSLEWIGRINPYNGDTFYNQKFKGKATLTVDKSSSTAHMELRSLTSEDSAVYYCARVLFLDFDDPYLMDYWGQGTSVTVSS

SEQ ID NO: 45 - protein, 482 aa, synthetic construct
MALPVTALLLPLALLLHAARPSSELTQDPAVSVALGQTVRITCRGDSLRKYYASWYQQKPRQAPQLVIYHKNNRASGIPDRFSGSISGNTASLTITGAQAEDEAAYFCNSRDTSGNYLVFGGGTKVTVLGGGGGSGGGGSGGGGSQLQLQESGPGLVKPSETLSLTCAVSGASISNWWSWVRQPPGKGLEWIGEVHHSGVTTYKPSLKSRVTISVDNSKNQLSLKLTSVTAADTAVYYCAREFADDAFDIWGRGTMVTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 238 aa, synthetic construct
SSELTQDPAVSVALGQTVRITCRGDSLRKYYASWYQQKPRQAPQLVIYHKNNRASGIPDRFSGSISGNTASLTITGAQAEDEAAYFCNSRDTSGNYLVFGGGTKVTVLGGGGGSGGGGSGGGGSQLQLQESGPGLVKPSETLSLTCAVSGASISNWWSWVRQPPGKGLEWIGEVHHSGVTTYKPSLKSRVTISVDNSKNQLSLKLTSVTAADTAVYYCAREFADDAFDIWGRGTMVTV

SEQ ID NO: 47 - protein, 109 aa, synthetic construct
SSELTQDPAVSVALGQTVRITCRGDSLRKYYASWYQQKPRQAPQLVIYHKNNRASGIPDRFSGSISGNTASLTITGAQAEDEAAYFCNSRDTSGNYLVFGGGTKVTVLG

SEQ ID NO: 48 - protein, 114 aa, synthetic construct
QLQLQESGPGLVKPSETLSLTCAVSGASISNWWSWVRQPPGKGLEWIGEVHHSGVTTYKPSLKSRVTISVDNSKNQLSLKLTSVTAADTAVYYCAREFADDAFDIWGRGTMVTV

SEQ ID NO: 49 - protein, 250 aa, synthetic construct
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSS

SEQ ID NO: 50 - protein, 250 aa, synthetic construct
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSS

SEQ ID NO: 51 - protein, 250 aa, synthetic construct
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSS

SEQ ID NO: 52 - protein, 250 aa, synthetic construct
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSS

SEQ ID NO: 53 - protein, 250 aa, synthetic construct
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSS

SEQ ID NO: 54 - protein, 250 aa, synthetic construct
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSS

SEQ ID NO: 55 - protein, 250 aa, synthetic construct
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSS

SEQ ID NO: 56 - protein, 250 aa, synthetic construct
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIKGGGGSGGGGSGGGGSEIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSS

SEQ ID NO: 57 - protein, 112 aa, synthetic construct
DIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIK

SEQ ID NO: 58 - protein, 112 aa, synthetic construct
DIVMTQSPLSLPVTPGEPASISCRSTKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 59 - protein, 123 aa, synthetic construct
EIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSS

SEQ ID NO: 60 - protein, 123 aa, synthetic construct
EIQLQQSGAEVKKPGSSVKVSCKASGYTFTDYDMHWVRQAPGQGLEWIGTIDPETGGTAYNQKFKGRATLTADRSTSTAYMELSSLRSEDTAVYYCTSFYYTYSNYDVGFAYWGQGTLVTVSS

SEQ ID NO: 61 - protein, 123 aa, synthetic construct
EIQLQQSGAEVKKPGSSVKVSCKASGYTFTDYDMHWVKQAPGQGLEWIGTIDPETGGTAYNQKFKGKATLTADRSTSTAYMELSSLRSEDTAVYYCTSFYYTYSNYDVGFAYWGQGTLVTVSS

SEQ ID NO: 62 - protein, 123 aa, synthetic construct
EIQLQQSGAEVKKPGSSVKVSCKASGYTFTDYDMHWVKQAPGHGLEWIGTIDPETGGTAYNQKFKGKATLTADRSTSTAYMELSSLTSEDTAVYYCTSFYYTYSNYDVGFAYWGQGTLVTVSS

SEQ ID NO: 63 - protein, 482 aa, synthetic construct
MALPVTALLLPLALLLHAARPDIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKGGGGSGGGGSGGGGSEVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 238 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKGGGGSGGGGSGGGGSEVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 65 - protein, 107 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 66 - protein, 116 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 67 - protein, 482 aa, synthetic construct
MALPVTALLLPLALLLHAARPDIQMTQSSSFLSVSLGGRVTITCKASDLIHNWLAWYQQKPGNAPRLLISGATSLETGVPSRFSGSGSGNDYTLSIASLQTEDAATYYCQQYWTTPFTFGSGTKVEIKGGGGSGGGGSGGGGSDVQLQESGPGLVNPSQSLSLTCTVTGYSITNDYAWNWIRQFPGNKLEWMGYINYSGYTTYNPSLKSRISITRDTSKNQFFLHLNSVTTEDTATYYCARWDGGLTYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 238 aa, synthetic construct
DIQMTQSSSFLSVSLGGRVTITCKASDLIHNWLAWYQQKPGNAPRLLISGATSLETGVPSRFSGSGSGNDYTLSIASLQTEDAATYYCQQYWTTPFTFGSGTKVEIKGGGGSGGGGSGGGGSDVQLQESGPGLVNPSQSLSLTCTVTGYSITNDYAWNWIRQFPGNKLEWMGYINYSGYTTYNPSLKSRISITRDTSKNQFFLHLNSVTTEDTATYYCARWDGGLTYWGQGTLVTVSA

SEQ ID NO: 69 - protein, 107 aa, synthetic construct
DIQMTQSSSFLSVSLGGRVTITCKASDLIHNWLAWYQQKPGNAPRLLISGATSLETGVPSRFSGSGSGNDYTLSIASLQTEDAATYYCQQYWTTPFTFGSGTKVEIK

SEQ ID NO: 70 - protein, 116 aa, synthetic construct
DVQLQESGPGLVNPSQSLSLTCTVTGYSITNDYAWNWIRQFPGNKLEWMGYINYSGYTTYNPSLKSRISITRDTSKNQFFLHLNSVTTEDTATYYCARWDGGLTYWGQGTLVTVSA

SEQ ID NO: 71 - protein, 298 aa, synthetic construct
MALPVTALLLPLALLLHAARPGTSLSPPPESSGSPQQPGLSAPHSRQIPAPQGAVLVQREKDLPNYNWNSFGLRFTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 54 aa, synthetic construct
GTSLSPPPESSGSPQQPGLSAPHSRQIPAPQGAVLVQREKDLPNYNWNSFGLRF

SEQ ID NO: 73 - DNA, 330 bp, synthetic construct
  source 1..330: mol_type = other DNA
cagagcgtgctgacacaacccgccagcgtgagcggcagccccggccaatccgtgacaatcagctgcaccggaaccagctccgatgtgggcggctacaactatgtcagctggtaccagcagcaccccggcaagccccctaagctcatgatctacgaggtcagcaagagaccctccggagtgcccgacagattctccggctccaaatccggcaacaccgcttccctcaccatcagcggactccaagccgaagatgaggccgactactactgctccagccacaccagcagcaacacactgatctttggcggcggcaccaaagtgacagtgctg

SEQ ID NO: 74 - DNA, 354 bp, synthetic construct
  source 1..354: mol_type = other DNA
gaagtgcagctggtgcaatccggcgctgaggtgaagaagcccggcgaatctctgaagattagctgcaaaggctccggctacaccttcacaaactactggatcggctgggtgagacagatgcccggcaagggactggagtggatgggaagaatctaccccagcgactcctacaccaactactccccctccttccaaggccaagtgaccattagcgccgacaagagcatctccaccgcctatctgcagtggtccagcctcaaggccagcgatacagccatgtactactgcgccagagacctcgagcctacccatcattacagctggggccaaggcacactcgtgacagtgagctcc

SEQ ID NO: 75 - DNA, 330 bp, synthetic construct
  source 1..330: mol_type = other DNA
ctccccgtgctgacacagcctccttccgcctccggaacacccggccagagagtgaccatcagctgcagcggctccagctccaacatcggcagcaacgccgtggactggtatcagcaactgcccggcacagctcccaagctgctgatctacaccaacaacagaaggcccagcggcgtgcccgatagattcagcggcagcaaatccggcacaagcggctctctggccatttccggactgcagttcgaggacgaggctgactactactgcgctgcttgggatgactctctgtccggcgtgatctttggcggaggcaccaaggtcacagtgctg

SEQ ID NO: 76 - DNA, 363 bp, synthetic construct
  source 1..363: mol_type = other DNA
caagtgcaactggtgcagagcggcgctgaggtgaagaagcccggcagcagcgtgaaggtcagctgcaaggccagcgaagacacattcagctcctacgtgatcagctgggtgagacaagctcccggccaaggactggagtggatgggagccttcatccctctgctgggcagagtgcactatgcccaagagttccaaggcagactgacaatcaccgccgacgagtccaccggcaccgcttacatggagctgaaaggactgaggagcgacgacaccgccatgtactactgcgccagagatcaaggcttcgccggcgatgacgccttcgatatctggggccaaggcacaatggtgacagtgagcagc

SEQ ID NO: 77 - DNA, 324 bp, synthetic construct
  source 1..324: mol_type = other DNA
cagcccgtgctgacccaagaccccgtggtgtccgtggctctgggacagaccgtgaggatcacatgccaaggcgattctctgaggagctactatgccacatggtaccagcagaaacccggactggcccccgtgagagtcatcttcggaaagaactctaggcccagcggcatccccgatagattcagcggcagcagcagcggcaataccgcctccctcaccatcaccggcgcccaagccgaggacgaagccgactactactgcaacagctgggacaacagcgccaaccatcccgtgttcggcggcggcacaaagctgacagtgctc

SEQ ID NO: 78 - DNA, 366 bp, synthetic construct
  source 1..366: mol_type = other DNA
gaggtgcagctcgtgcagtccggcgccgaagtgaggaagcccggcagctccgtgaaggtgagctgtaaggccagcggcgacaacttcaacaactacggcatcacatgggtgagacaagcccccggccaaggactggagtggatgggaagactgatcaccgtgctgggactggacaactacgcccagaagttccaaggaagactgacaatcaccgctgacgagtccaccggcaccgcctatatggaactgaccggactggagcccgaggataccgccgtgtactattgcgctagagacatcatgtcccccgaggccatcgatgccttcgacgtgtggggacaaggcacactggtgaccgtgagctcc

SEQ ID NO: 79 - DNA, 321 bp, synthetic construct
  source 1..321: mol_type = other DNA
gacatccagatgacacagagcccttcctctctgagcgcctccgtcggagatagagtgaccatcacatgcagagcctcccaagacgtcagcaactatctgaactggtaccagcagaagcccggcaaggcccctaaactgctgatctatggcgccagcaatagacagaccggcgtgccttccagattctccggcagcggaagcggcaccgatttcacactgacaatttcctctctgcagcccgaggactttgccacctactactgccagcaagaggacagattccctacaaccttcggccaaggcacaaaggtggagatcaaa

SEQ ID NO: 80 - DNA, 366 bp, synthetic construct
  source 1..366: mol_type = other DNA
gaagtgcagctggtgcagagcggagctgaggtgagaaagcccggcagcagcgtcaaggtgagctgtaaggccagcggcgacaacttcaacaactacggcatcacatgggtgaggcaagcccccggccaaggcctcgagtggatgggaagactgatcaccgtgctcggactggacaactacgcccagaagttccaaggcagactgacaatcaccgccgatgagtccaccggaacagcctacatggagctgaccggactggagcccgaagacacagctgtgtactactgcgctagagacatcatgtcccccgaggccatcgatgcctttgacgtgtggggccaaggcacactggtgacagtcagcagc

SEQ ID NO: 81 - DNA, 45 bp, synthetic construct
  source 1..45: mol_type = other DNA
ggaggaggaggcagcggaggcggaggcagcggaggaggcggcagc

SEQ ID NO: 82 - protein, 110 aa, synthetic construct
QSVLTQPASVSGSPGQSVTISCTGTSSDVGGYNYVSWYQQHPGKPPKLMIYEVSKRPSGVPDRFSGSKSGNTASLTISGLQAEDEADYYCSSHTSSNTLIFGGGTKVTVL

SEQ ID NO: 83 - protein, 118 aa, synthetic construct
EVQLVQSGAEVKKPGESLKISCKGSGYTFTNYWIGWVRQMPGKGLEWMGRIYPSDSYTNYSPSFQGQVTISADKSISTAYLQWSSLKASDTAMYYCARDLEPTHHYSWGQGTLVTVSS

SEQ ID NO: 84 - protein, 110 aa, synthetic construct
LPVLTQPPSASGTPGQRVTISCSGSSSNIGSNAVDWYQQLPGTAPKLLIYTNNRRPSGVPDRFSGSKSGTSGSLAISGLQFEDEADYYCAAWDDSLSGVIFGGGTKVTVL

SEQ ID NO: 85 - protein, 121 aa, synthetic construct
QVQLVQSGAEVKKPGSSVKVSCKASEDTFSSYVISWVRQAPGQGLEWMGAFIPLLGRVHYAQEFQGRLTITADESTGTAYMELKGLRSDDTAMYYCARDQGFAGDDAFDIWGQGTMVTVSS

SEQ ID NO: 86 - protein, 108 aa, synthetic construct
QPVLTQDPVVSVALGQTVRITCQGDSLRSYYATWYQQKPGLAPVRVIFGKNSRPSGIPDRFSGSSSGNTASLTITGAQAEDEADYYCNSWDNSANHPVFGGGTKLTVL

SEQ ID NO: 87 - protein, 122 aa, synthetic construct
EVQLVQSGAEVRKPGSSVKVSCKASGDNFNNYGITWVRQAPGQGLEWMGRLITVLGLDNYAQKFQGRLTITADESTGTAYMELTGLEPEDTAVYYCARDIMSPEAIDAFDVWGQGTLVTVSS

SEQ ID NO: 88 - protein, 107 aa, synthetic construct
DIQMTQSPSSLSASVGDRVTITCRASQDVSNYLNWYQQKPGKAPKLLIYGASNRQTGVPSRFSGSGSGTDFTLTISSLQPEDFATYYCQQEDRFPTTFGQGTKVEIK

SEQ ID NO: 89 - protein, 122 aa, synthetic construct; identical to SEQ ID NO: 87

SEQ ID NO: 90 - protein, 15 aa, synthetic construct
GGGGSGGGGSGGGGS

SEQ ID NO: 91 - DNA, 717 bp, synthetic construct
  source 1..717: mol_type = other DNA
gaggtccaactgcagcagtctggagctgagctgatgaagcctgggacttcagtgaagatgtcctgcaaggctgctggatacaccttcactaagtactggataggttgggtaaagcagaggcctggacatggccttgagtggattggagatatttaccctggaggtggttatactctcttcaatgagaagttcaagggcaaggccacactgactgcagacacatcctccagcacagcctacatgcagctcagcagcctgacatctgaggactctgccatctattactgtgcaagagggaactactggggccaaggcaccactctcaccgtctcctcaggtggtggtggtagcggcggcggcggctctggtggtggtggatccgatgttgtgatgacccaaactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcacacgttcggagggggcaccaagctggaaatgaaa

SEQ ID NO: 92 - protein, 239 aa, synthetic construct
EVQLQQSGAELMKPGTSVKMSCKAAGYTFTKYWIGWVKQRPGHGLEWIGDIYPGGGYTLFNEKFKGKATLTADTSSSTAYMQLSSLTSEDSAIYYCARGNYWGQGTTLTVSSGGGGSGGGGSGGGGSDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEMK

SEQ ID NO: 93 - protein, 112 aa, synthetic construct
EVQLQQSGAELMKPGTSVKMSCKAAGYTFTKYWIGWVKQRPGHGLEWIGDIYPGGGYTLFNEKFKGKATLTADTSSSTAYMQLSSLTSEDSAIYYCARGNYWGQGTTLTVSS

SEQ ID NO: 94 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPHTFGGGTKLEMK

SEQ ID NO: 95 - DNA, 708 bp, synthetic construct
  source 1..708: mol_type = other DNA
caggtcaagctgcaacagtctgggcccgagctggtgaggcctggggcttcagtgaagatgtcctgcaaggcttcaggctataccttcaccacctactggatgcactgggtgaaacagaggccaggacaaggccttgagtggattggcatgattgatccctccaatagtgacactaggttaaatcagaagttcaaggacaaggccacattgaatgttgacacatcctccaacacagcctacatgcacctcagcagcctgacatctgaggactctgcagtctattactgtacattaggtgggactgagtattggggccaaggcgccactctcacagtctcctcaggtggtggtggtagcggcggcggcggctctggtggtggtggatccagtattgtgatgacccagtctccagccaccctgtctgtgactccaggagatagagtctctctttcctgcagggccagccagagtattggcgactacctacactggtttcaacaaaaatcacatgagtctccaaggcttctcatcaaatatgcttcccaatccatctctgggatcccctccaggttcagtggcagtggatcagggtcagatttcactctcattatcaacactatggaacctgaagatgttggagtgtattactgtcaaaatgctcacacctatccgtacacgttcggagggggcaccaagctggaaatcaaa

SEQ ID NO: 96 - protein, 236 aa, synthetic construct
QVKLQQSGPELVRPGASVKMSCKASGYTFTTYWMHWVKQRPGQGLEWIGMIDPSNSDTRLNQKFKDKATLNVDTSSNTAYMHLSSLTSEDSAVYYCTLGGTEYWGQGATLTVSSGGGGSGGGGSGGGGSSIVMTQSPATLSVTPGDRVSLSCRASQSIGDYLHWFQQKSHESPRLLIKYASQSISGIPSRFSGSGSGSDFTLIINTMEPEDVGVYYCQNAHTYPYTFGGGTKLEIK

SEQ ID NO: 97 - protein, 114 aa, synthetic construct
QVKLQQSGPELVRPGASVKMSCKASGYTFTTYWMHWVKQRPGQGLEWIGMIDPSNSDTRLNQKFKDKATLNVDTSSNTAYMHLSSLTSEDSAVYYCTLGGTEYWGQGATLTVSS

SEQ ID NO: 98 - protein, 107 aa, synthetic construct
SIVMTQSPATLSVTPGDRVSLSCRASQSIGDYLHWFQQKSHESPRLLIKYASQSISGIPSRFSGSGSGSDFTLIINTMEPEDVGVYYCQNAHTYPYTFGGGTKLEIK

SEQ ID NO: 99 - DNA, 738 bp, synthetic construct
  source 1..738: mol_type = other DNA
gaagtgaagctgaagcagtctggggctgaactggtgaagcctggggcttcagtgaagttgtcctgcaaggcttctggctacaccctcaccagttactatatttactgggtgaagcagaggcctggacaaggccttgagtggattggggggattagtcctagcaatggtaatacttacttcactgagaagttcaagaacatggccacactgactgtagacaaatcctccaacacagcctacatgcaactcagcagcctgacatctgaggactctgcggtctattactgtgcaagaaactatgattacgacgtggggtttgcttactggggccaagggactctggtcacagtctcctcaggtggtggtggtagcggcggcggcggctctggtggtggtggatccgatgttgtgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggaccaagctggaaatcaaa

SEQ ID NO: 100 - protein, 246 aa, synthetic construct
EVKLKQSGAELVKPGASVKLSCKASGYTLTSYYIYWVKQRPGQGLEWIGGISPSNGNTYFTEKFKNMATLTVDKSSNTAYMQLSSLTSEDSAVYYCARNYDYDVGFAYWGQGTLVTVSSGGGGSGGGGSGGGGSDVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 101 - protein, 119 aa, synthetic construct
EVKLKQSGAELVKPGASVKLSCKASGYTLTSYYIYWVKQRPGQGLEWIGGISPSNGNTYFTEKFKNMATLTVDKSSNTAYMQLSSLTSEDSAVYYCARNYDYDVGFAYWGQGTLVTVSS

SEQ ID NO: 102 - protein, 112 aa, synthetic construct
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 103 - DNA, 726 bp, synthetic construct
  source 1..726: mol_type = other DNA
caggtgcagctgaaggagtctgggggaggcttagtgcagcctggggagtccctgaaactctcctgtgaatccagtgaattcccattcccttcccatgacatgtcttgggtccgcaagactccggagaagaggctggagttggtcgcagccattaatagtgatggtggtagcacctactatccagacaccatggagagacgattcatcatctccagagacaataccaagaagaccctgtacctgcaaatgagcagtctgaggtctgaggacacagccttgtattactgtgcaagcctacccacgtttgcttactggggccaagggactctggtcactgtctctgcaggtggtggtggtagcggcggcggcggctctggtggtggtggatccagtattgtgatgacccaaactccactcactttgtcggttaccattggacaaccagcctctatctcttgcaagtcaagtcagagcctcttatatagtgatggacaaacctatttgaattggttattacagaggccaggccagtctccaaagcgcctaatctatctggtgtctaaactggactctggagtccctgacaggttcactggcagtggatcaggtacagattttacactgaaaatcagcagagtggaggctgaggatttgggagtttattactgcgtgcaaactacacattttccgtacacgttcggaggggggaccaagctggaaatgaaa

SEQ ID NO: 104 - protein, 242 aa, synthetic construct
QVQLKESGGGLVQPGESLKLSCESSEFPFPSHDMSWVRKTPEKRLELVAAINSDGGSTYYPDTMERRFIISRDNTKKTLYLQMSSLRSEDTALYYCASLPTFAYWGQGTLVTVSAGGGGSGGGGSGGGGSSIVMTQTPLTLSVTIGQPASISCKSSQSLLYSDGQTYLNWLLQRPGQSPKRLIYLVSKLDSGVPDRFTGSGSGTDFTLKISRVEAEDLGVYYCVQTTHFPYTFGGGTKLEMK

SEQ ID NO: 105 - protein, 115 aa, synthetic construct
QVQLKESGGGLVQPGESLKLSCESSEFPFPSHDMSWVRKTPEKRLELVAAINSDGGSTYYPDTMERRFIISRDNTKKTLYLQMSSLRSEDTALYYCASLPTFAYWGQGTLVTVSA

SEQ ID NO: 106 - protein, 112 aa, synthetic construct
SIVMTQTPLTLSVTIGQPASISCKSSQSLLYSDGQTYLNWLLQRPGQSPKRLIYLVSKLDSGVPDRFTGSGSGTDFTLKISRVEAEDLGVYYCVQTTHFPYTFGGGTKLEMK

SEQ ID NO: 107 - DNA, 720 bp, synthetic construct
  source 1..720: mol_type = other DNA
caggttcagctgcagcagtctggggcagagcttgtgaggtcaggggcctcagtcaagttgtcctgcacagattctggcttcaacattaaagactactatatgaactgggtgaaacagcggcctgaacagggcctggagtggattggatggattgatcctgaaaatggtgttactcaatatgacccgaagttccagggcaaggccactgtgactgcagacacattctccaacacagcctacctgcagctcagcagcctgacatctgaggacactgccgtctattactgtaatgcaaactataagtacgactggtttggttactggggccaagggactctggtcactgtctctgcaggtggtggtggtagcggcggcggcggctctggtggtggtggatccaacattgtaatgacccagtctccatcctccttatctgcctctctgggagaaagagtcagtctcacttgtcgggcaagtcaggaaattagtggttacttaagctggcttcagcagaaaccagatggaactattaaacgcctgatctacgccgcatccactttagattctggtgtcccagaaaggttcagtggcagtaggtctgggtcagattattctctcaccatcagcagccttgagtctgaagattttgcagactattactgtctacaatatgctagttatccgtggacgttcggtggagggaccaagctggaaatgaaa

SEQ ID NO: 108 - protein, 240 aa, synthetic construct
QVQLQQSGAELVRSGASVKLSCTDSGFNIKDYYMNWVKQRPEQGLEWIGWIDPENGVTQYDPKFQGKATVTADTFSNTAYLQLSSLTSEDTAVYYCNANYKYDWFGYWGQGTLVTVSAGGGGSGGGGSGGGGSNIVMTQSPSSLSASLGERVSLTCRASQEISGYLSWLQQKPDGTIKRLIYAASTLDSGVPERFSGSRSGSDYSLTISSLESEDFADYYCLQYASYPWTFGGGTKLEMK

SEQ ID NO: 109 - protein, 118 aa, synthetic construct
QVQLQQSGAELVRSGASVKLSCTDSGFNIKDYYMNWVKQRPEQGLEWIGWIDPENGVTQYDPKFQGKATVTADTFSNTAYLQLSSLTSEDTAVYYCNANYKYDWFGYWGQGTLVTVSA

SEQ ID NO: 110 - protein, 107 aa, synthetic construct
NIVMTQSPSSLSASLGERVSLTCRASQEISGYLSWLQQKPDGTIKRLIYAASTLDSGVPERFSGSRSGSDYSLTISSLESEDFADYYCLQYASYPWTFGGGTKLEMK

SEQ ID NO: 111 - DNA, 723 bp, synthetic construct
  source 1..723: mol_type = other DNA
caggtgcagctgaagcagtctgggcctgggctggtgaggcctggggcttcagtgacgatgtcctgcaaggcttcaggctataccttcaccagcaactggatgcactgggttaaacagaggcctggacaaggccttgattggattggcatgattgatccttccaatagtgacactaggttaaatcagaagttcaaggacaaggccacattgaatgtagacaaatcctccaacacagcctacatgcagctcagcagcctgacgtctgatgactctgcagtctattactgtatgagagggggcggcgactactggggccaaggcaccactctcacagtctcctcaggtggtggtggtagcggcggcggctctggtggtggtggatccgacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgtacacgttcggaggggggaccaagctggaaatgaaa

SEQ ID NO: 112 - protein, 242 aa, synthetic construct
QVQLKQSGPGLVRPGASVTMSCKASGYTFTSNWMHWVKQRPGQGLDWIGMIDPSNSDTRLNQKFKDKATLNVDKSSNTAYMQLSSLTSDDSAVYYCMRGGGDYWGQGTTLTVSSGGGGSGGGGSGGGGSDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPYTFGGGTKLEMK

SEQ ID NO: 113 - protein, 114 aa, synthetic construct
QVQLKQSGPGLVRPGASVTMSCKASGYTFTSNWMHWVKQRPGQGLDWIGMIDPSNSDTRLNQKFKDKATLNVDKSSNTAYMQLSSLTSDDSAVYYCMRGGGDYWGQGTTLTVSS

SEQ ID NO: 114 - protein, 113 aa, synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPYTFGGGTKLEMK

SEQ ID NO: 115 - DNA, 720 bp, synthetic construct
  source 1..720: mol_type = other DNA
gaggtgcagctgaaggagtctggggcagagcttgtgaggtcaggggcctcagtcaagttgtcctgcacagcttctgccttcaacattaaagactactatatgaactggatgaagcagaggcctgaacagggcctggagtggattggatggattgatcctgagaatggtgatactaaatatgccccgaagttccagggcaaggccactatcactgcagacacatcctccagcacagcctacctgcagctcaccagcctgacatctgaggactctgccgtcttctattgtaatgtaaactataagtacgactggtttgcttactggggccaagggactctggtcactgtctctgcaggtggtggtggtagcggcggcggcggctctggtggtggtggatccgatgttgtgatgacccagtcccaaaaattcatgtccacatcagtaggagacagggtcagcgtcacctgcaaggccagtcagaatgtgggtactaatgtagcctggtatcaacagaaaccagggcaatctcctagagcactgatttactcggcatcctaccggtacagtggagtccctgatcgcttcacaggcagtggatctgggacagatttcactctcaccatcagcaatgtgcagtctgaagacttggcagagtatttctgtcagcaatataacacctatccgtggacgttcggtggggggacaaagctggaaataaaa

SEQ ID NO: 116 - protein, 240 aa, synthetic construct
EVQLKESGAELVRSGASVKLSCTASAFNIKDYYMNWMKQRPEQGLEWIGWIDPENGDTKYAPKFQGKATITADTSSSTAYLQLTSLTSEDSAVFYCNVNYKYDWFAYWGQGTLVTVSAGGGGSGGGGSGGGGSDVVMTQSQKFMSTSVGDRVSVTCKASQNVGTNVAWYQQKPGQSPRALIYSASYRYSGVPDRFTGSGSGTDFTLTISNVQSEDLAEYFCQQYNTYPWTFGGGTKLEIK

SEQ ID NO: 117 - protein, 118 aa, synthetic construct
EVQLKESGAELVRSGASVKLSCTASAFNIKDYYMNWMKQRPEQGLEWIGWIDPENGDTKYAPKFQGKATITADTSSSTAYLQLTSLTSEDSAVFYCNVNYKYDWFAYWGQGTLVTVSA

SEQ ID NO: 118 - protein, 107 aa, synthetic construct
DVVMTQSQKFMSTSVGDRVSVTCKASQNVGTNVAWYQQKPGQSPRALIYSASYRYSGVPDRFTGSGSGTDFTLTISNVQSEDLAEYFCQQYNTYPWTFGGGTKLEIK

SEQ ID NO: 119 - DNA, 720 bp, synthetic construct
  source 1..720: mol_type = other DNA
caggtccaactgcagcagtctgggcctgagctggtgaggcctggggcttcagtgacgatgtcctgcaaggcttcaggctataccttcaccagcaactggatgcactgggttaaacagaggcctggacaaggccttgattggattggcatgattgatccttccaatagtgacactaggttaaatcagaagttcaaggacaaggccacattgaatgtagacaaatcctccaacacagcctacatgcagctcagcagcctgacgtctgaggactctgcagtctattactgtatgagagggggcggcgactactggggccaaggcaccactctcacagtctcctcaggtggtggtggtagcggcggcggcggctctggtggtggtggatccgacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcacagttgggagattccgtacacgttcggagggggcaccaagctggaaatgaaa

SEQ ID NO: 120 - protein, 240 aa, synthetic construct
QVQLQQSGPELVRPGASVTMSCKASGYTFTSNWMHWVKQRPGQGLDWIGMIDPSNSDTRLNQKFKDKATLNVDKSSNTAYMQLSSLTSEDSAVYYCMRGGGDYWGQGTTLTVSSGGGGSGGGGSGGGGSDIVMTQSPASLAVSLGQRATISCKASQSVDYDGDSYMNWYQQKPGQPPKLLIYAASNLESGIPARFSGSGSGTDFTLNIHPVEEEDAATYYCQHSWEIPYTFGGGTKLEMK

SEQ ID NO: 121 - protein, 114 aa, synthetic construct
QVQLQQSGPELVRPGASVTMSCKASGYTFTSNWMHWVKQRPGQGLDWIGMIDPSNSDTRLNQKFKDKATLNVDKSSNTAYMQLSSLTSEDSAVYYCMRGGGDYWGQGTTLTVSS

SEQ ID NO: 122 - protein, 111 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQHSWEIPYTFGGGTKLEMK

SEQ ID NO: 123 - protein, 499 aa, synthetic construct
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSDIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 124 - protein, 501 aa, synthetic construct
EVKLVESGGGLVQPGGSMKLSCVASGFTFSNYWMNWVRQSPEKGLEWVAEIRLKSNNYTTHYAESVKGRFTISRDDSKSSVSLQMNNLRVEDTGIYYCTRHYYFDYWGQGTTLTVSSGGGGSGGGGSGGGGSDIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKRASGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 125 - protein, 492 aa, synthetic construct
EVQLQQSGAELVKPGASVKLSCTASGFNINDTYMHWVKQRPEQGLEWIGRIDPANGNTKYDPKFQGKATITADTSSNTAYLQLSSLTSEDTAVYYCARGARGSRFAYWGQGTLVTVSAGGGGSGGGGSGGGGSDIQMTQSPASLSVSVGETVTITCRASENIYSNLAWYQQKQGKSPQLLVYVATNLADGVPSRFSGSGSGTQYSLKINSLQSEDFGSYYCQHFWGTPYTFGGGTKLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 126 - protein, 499 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 127 - protein, 507 aa, synthetic construct
EVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSSGGGGSGGGGSGGGGSDIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRASGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 128 - protein, 498 aa, synthetic construct
QVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSSGGGGSGGGGSGGGGSDVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 129 - protein, 496 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSGGGGSGGGGSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 130 - protein, 501 aa, synthetic construct
QVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSAGGGGSGGGGSGGGGSDVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRASGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 131 - protein, 501 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 132 - protein, 496 aa, synthetic construct
EVQLQQSGPELVKPGGSMKISCKASGYSFTGYTMNWVKQSHGKNLEWIGLINPYNGGISYNQKFKDKATLTMDKSSSTAYMELLSLTSEDSAIYFCARDYRYEGFDYWGQGTLVTVSAGGGGSGGGGSGGGGSQIVLTQSPAIMSASLGERVTMTCTASSVVISTYLHWYQQKPGSSPKLWIYSTSNLASGVPVRFSGSGSGTSYSLTISSMEAEDAATYYCHQYHRSPWTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 133 - protein, 505 aa, synthetic construct
EIQLQQSGAEVKKPGSSVKVSCKASGYTFTDYDMHWVRQAPGQGLEWMGTIDPETGGTAYNQKFKGRVTITADKSTSTAYMELSSLRSEDTAVYYCTSFYYTYSNYDVGFAYWGQGTLVTVSSGGGGSGGGGSGGGGSDIVMTQSPLSLPVTPGEPASISCRSTKSLLHSNGNTYLYWYLQKPGQSPQLLIYRMSNLASGVPDRFSGSGSGTDFTLKISRVEAEDVGVYYCMQHLEYPFTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 134 - protein, 493 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSGGGGSGGGGSGGGGSDIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 135 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 244 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGGGGSGGGGSGGGGSEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSS

SEQ ID NO: 137 - protein, 252 aa, synthetic construct
DIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRAGGGGSGGGGSGGGGSEVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSS

SEQ ID NO: 138 - protein, 243 aa, synthetic construct
DVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIKGGGGSGGGGSGGGGSQVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSS

SEQ ID NO: 139 - protein, 241 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 140 - protein, 246 aa, synthetic construct
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRAGGGGSGGGGSGGGGSQVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSA

SEQ ID NO: 141 - protein, 199 aa, synthetic construct
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 142 - protein, 240 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 143 - protein, 281 aa, synthetic construct
MNVPTQLLGLLLLWLTGGKCDIQMTQSPASLSASLEEIVTITCKASQAIDAYLSWYQQKPGKSPQLLIYDATSLADGVPSRFSGSRSGTQYSLKISRPQVDDSGIYYCLQSYSTPFTFGSGTKLEIKGGGGGSGGGGSGGGGSMAVLVLLLCLLIFPSCVLSQVQLKESGPGLVQPSQTLSLTCTVSGSSLTSNSVSWIRQPPGKGLEWMGVIWSNGDADYNSAIKSRLSISRDTSKSQVFLKMNSLQTEDTAMYFCASPYYGYYFPFDYWGQGVMVTVSS

SEQ ID NO: 144 - protein, 285 aa, synthetic construct
METDTLLLWVLLLWVPGSTGDIVLTQSPALAVSLGQRATISCRASQSVSISSHDLMQWYQQKPGQQPKLLIYDAFNLASGIPVRFSGSGSGTDFTLTIDPVQADDIATYYCQQSKDDPYTFGAGTKLELKGGGGGSGGGGSGGGGSMDIRLSLAFLVLFIKGVQCEVQLVESGGGLVQPGRSMKLSCAASGFTFSNYGMAWVRQAPTKGLEWVATISYDGSITYYRDSVKGRFTISRDHAKSTLYLQMNSLRSEDTATYYCTREEQYSSWYFDFWGPGIMVTVSS

SEQ ID NO: 145 - protein, 242 aa, synthetic construct
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIKGGGGGSGGGGSGGGGSQVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 146 - protein, 246 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 147 - protein, 255 aa, synthetic construct
MADYKDIVMTQSPSSVSASVGDRVTITCRASQNVDSAVAWYQQKPGKAPKALIYSASYRYSGVPSRFSGRGSGTDFTLTISSLQPEDFATYYCQQYYSTPWTFGQGTKVEIKRGGGGSGGGGSGGGGSEVKLVESGGGLVQPGRSLRLSCTASGFTFTDYYMSWVRQAPGKGLEWVGLIRSKADGYTTEYSASVKGRFTISRDDSKSILYLQMNSLKTEDTAVYYCARDAAYYSYYSPEGAMDYWGQGTLVTVSS

SEQ ID NO: 148 - protein, 244 aa, synthetic construct
QSVLTQPPSASGTPGQRVTISCSGSSSNIGINTVNWYQQLPGTAPKLLIYDNNKRPSGVPDRFSGSKSGTSASLAISGLRSEDEADYYCATWDDSLSGWVFGGGTKLTVLGGGGSGGGGSGGGGSEVQLLESGGGLVQPGGSLRLSCAASGFTFSDYYMSWIRQAPGKGLEWVSAISGSGGSTYYADSVKGRFTISRDNSKNTLYLQMNSLRAEDTAVYYCAKEDWEGASFDYWGQGTLVTVSS

SEQ ID NO: 149 - protein, 245 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRGGGGSGGGGSGGGGSQVQLVQSGAEVKKPGASVKVSCKASGYTFTNYGMNWVRQAPGQGLEWMGWINTYTGEPTYADAFKGRVTMTTDTSTSTAYMELRSLRSDDTAVYYCARDYGDYGMDYWGQGTTVTVSS

SEQ ID NO: 150 - protein, 263 aa, synthetic construct
AQAAELDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSSAKTTPPSVTSGQAGQ

SEQ ID NO: 151 - protein, 239 aa, synthetic construct
EVQLLESGGGLVQPGGSLRLSCAASGFTFSSYAMSWVRQAPGKGLEWVSTIQKQGLPTEYADSVKGRFTISRDNSKNTLYLQMNSLRAEDTAVYYCAKNRAKFDYWGQGTLVTVSSGGGGSGGGGSGGGGSTDIQMTQSPSSLSASVGDRVTITCRASQSISSYLNWYQQKPGKAPKLLIYNASMLQSGVPSRFSGSGSGTDFTLTISSLQPEDFATYYCQQNRGFPLTFGQGTKVEIK

SEQ ID NO: 152 - protein, 239 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 153 - protein, 246 aa, synthetic construct
STVMTQSPSFLSASVGDRVTITCKASQSVSNDVAWYQQKPCQSPKVLIYFASNRYTGVPDRFSGSGYGTDFTLTISSLQAEDVAVYFCQQDYSSPLTFGAGTKLEIKGGGGSGGGGSGGGGSQIQLVQSGSELKKPGASVKVSCKASGYTFTNFFVNWVKQAPGKGLKWMGWINTYTGEPSYADDFKGRFAFSLDASASTAYLQISSLKAEDMATYFCTRRTNYYGTSYYYAMDYWGQGTTVTVSS

SEQ ID NO: 154 - protein, 458 aa, synthetic construct
RIVMTQSPGTLSVSPGETATLSCRASQSFSNMLAWYQQKSGQPPRLLIYGVSTRAAGVPARFSGSGSGTEFTLTISNLQSEDFAVYYCQQYGDWPRYTFGQGTKVERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGGGVVRPGGSLRLPCAASGFTFDDYGMSWVRQAPGKGLEWVSGINWNGGSTGYADSVKGRFTISRDNAKNSLYLQMNSLRAEDTALYHCARGGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAG

SEQ ID NO: 155 - protein, 239 aa, synthetic construct
EIVLTQSPGTLSLSPGERATLSCRASQSVSSSYLAWYQQKPGQAPRLLIYGASSRATGIPDRFSGSGSGTDFTLTISSLEPEDFAVYYCQQYGSSPWTFGQGTKVEIKGGGGSGGGGSGGGGSEVQLVESGGGLVQPGGSLRLSCVASGFTFSNSWMSWVRQAPGKGLEWVANINEDGSEKFYVDSVKGRFTFSRDNAENSLYLQMNSLRAEDTAVYYCARVHWYFHLWGRGTLVTVSS

SEQ ID NO: 156 - protein, 242 aa, synthetic construct; identical to SEQ ID NO: 145

SEQ ID NO: 157 - protein, 679 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 246 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTGGGGSGGGGSGGGGSQVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFYDYVEAMDYWGQGTTVTVSS

SEQ ID NO: 159 - protein, 392 aa, synthetic construct
SRAATMETDTLLLWVLLLWVPGSTGDIQLTQSPSSLSASVGDRVTITCSASSSVRFIHWYQQKPGKAPKRLIYDTSKLASGVPSRPSGSGSGTDFTLTISSLQPEDFATYYCQQWSSSPFTFGQGTKVEIKGSTSGGGSGGGSGGGGSSEVQLVESGGGLVQPGGSLRLSCAASGFNIKDYYIHWVRQAPGKGLEWVAWIDPENGDTEFVPKFQGRATISADTSKNTAYLQMNSLRAEDTAVYYCKTGGFWGQGTLVTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 243 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSYPLTFGGGTKVEIKGGGGSGGGGSGGGGSQVQLVQSGAEVKKPGASVKVSCKASGYTFTDHAIHWVRQAPGQRLEWMGYFSPGNDDFKYSQKFQGRVTITADKSASTAYMELSSLRSEDTAVYYCARSLNMAYWGQGTLVTVSS

SEQ ID NO: 161 - protein, 243 aa, synthetic construct
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIKGGGGSGGGGSGGGGSQVQLVQSGAEVKKPGSSVKVSCKAFGGTFSSYAISWVRQAPGQGLEWMGRIIRFLGKTNHAQKFQGRVTLTADKSTNTAYMELSSLRSEDTAVYYCAGEPGDRDPDAVDIWGQGTMVTVSS

SEQ ID NO: 162 - protein, 249 aa, synthetic construct
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKRGGGGSGGGGSGGGGSQVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSSA

SEQ ID NO: 163 - protein, 237 aa, synthetic construct
DIKMTQSPSSMYASLGERVTITCKASQDINNYLSWFQQKPGKSPKTLIYRANRLVDGVPSRFSGSGSGQDYSLTISSLEYEDMGIYYCLKYDEFPYTFGGGTKLEIKGGGGSGGGGSGGGGSDVKLVESGGGLVKPGGSLKLSCAASGFTFSSYTMSWVRQTPEKRLEWVATISSGGTYTYYPDSVKGRFTISRDNAKNTLYLQMSSLKSEDTAMYYCTREAIFTYWGQGTLVTVSA

SEQ ID NO: 164 - protein, 239 aa, synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSS

SEQ ID NO: 165 - protein, 441 aa, synthetic construct
EIVMTQTPATLSVSAGERVTITCKASQSVSNHVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCYELLAENNFYPREAKVQWKVDNALQSGNSQESVTEQDSKDSTYSLSSTLTLSKADYEKHKVYACEVTHQGLSSPVTKSFNRGECGGGGSGGGGSGGGGSKIVMTQTPATLSVSAGERVTITCKASQSVSNHVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 685 aa, synthetic construct
DIQMTQSPSSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKDIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGTTVTVSSDIPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 239 aa, synthetic construct
EIVMTQTPLILSVTIGQPASISCKSSQSVLYSNGKTYLNWLLQRPGQSPKRLIYLVSKLDSGVPDRFTGSGSGTDFTLKISRVEAEDLGVYYCVQGSHFPYTFGGGTKLEIKRADAGGGGSGGGGSGGGGSGPELKKPGETVKIYCKASGYSFRDYSVHWVKQAPGKGLKWMGWINTETGEPTYVDEFKGRFAFFLEASANTVYLQISNLKNEDTATYFCDYRFTYWGQGTLVTVSAAK

SEQ ID NO: 168 - protein, 243 aa, synthetic construct
DIQMTQSPSSLSASVGDRVTITCRASQGISSWLAWYQQKPEKAPKSLIYAASSLQSGVPSRESGSGSGTDFTLTISSLQPEDEATYYCQQYNGYPYTEGQGTKLEIKGGGGSGGGGSGGGGSEVQLVESGGGLVKPGGSLRLSCAASGFTFSSYSMNWVRQAPGKGLEWVSSITSSSSYIYYADSVKGRFTISRDNAKNSLYLQMNSLRAEDTAVYYCARDRRYFDWFPIDYRGQGTLVTVSS

SEQ ID NO: 169 - protein, 285 aa, synthetic construct
MRVPAQLLGLLLLCFPGARCDIQMTQSPSSLSASVGDRVTITCRASQGITNYLAWFQQKPGKAPKSLIYAASSLQSGVPSKFSGSGSGTDFSLTISSLQPEDFATYYCQQYNSYPITFGQGTRLEIKGGGGSGGGGSGGGGSMELGLRWVLLVALLRGVQCQVQLVESGGGVVQPGRSLRLSCAASGFTFSNYVMHWVRQAPGKGLEWVAIIWYDGSNKYYADSVKGRFTISRDNSKNTLYLQMNSLRAEDTAVYYCAGGYNWNYEYHYYGMDVWGQGTTVTVSS

SEQ ID NO: 170 - protein, 279 aa, synthetic construct
MDFQVQIFSFLLISASVILSSGQIGLTQSPAIMSASPGEKVTMTCSASSSISYMHWYQQKPGTSPKTWIYDTSKLASGVPARFSGSGSGTSYSLTISSMEAEDAATYYCHQRSSHHTFGGGTKLEIKGGGGSGGGGSGGGGSMGWLWNLLFLMAAAQSAQAQIQLVQSGPELKKPGETVKISCKASGYTFTTYGMSWVKQAPGRGLKWMGWINPYSGVPTYAVDFKGRFAFSLETSASTAYLQINNLKNEDTATYFCARGGRRGDFGYWGQGTTLTVSS

SEQ ID NO: 171 - protein, 244 aa, synthetic construct
DIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGGGGSGGGGSGGGGSQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 172 - protein, 254 aa, synthetic construct
QVQLEQSGLGLVKPSQTLSLTCAISGDTVSSDSAAWNWIRQSPSRGLEWLGRTYYRSKWFNDYAVSVKGRITINSDTSKNQFSLQLNSVTPEDTAVYYCARSNSYYYYAMDVWGQGTLVTVSSGGGGSGGGGSGGGGSQAVLTQPSSLSASPGASASLTCTLRSGINVGIYRIYWYQQRPGSPPQILLTYKSDSDKYQGSGVPSRFSGSKDASANAGILLISGLQSEDEADYYCMIWHSGGWVFGGGTKVTVLG

SEQ ID NO: 173 - protein, 264 aa, synthetic construct
MALPVTALLLPLALLLHAARPQSVLTQPASVSGSPGQSVTISCTGTSSDVGGYNYVSWYQQHPGKPPKLMIYEVSKRPSGVPDRFSGSKSGNTASLTISGLQAEDEADYYCSSHTSSNTLIFGGGTKVTVLGGGGSGGGGSGGGGSEVQLVQSGAEVKKPGESLKISCKGSGYTFTNYWIGWVRQMPGKGLEWMGRIYPSDSYTNYSPSFQGQVTISADKSISTAYLQWSSLKASDTAMYYCARDLEPTHHYSWGQGTLVTVSS

SEQ ID NO: 174 - protein, 267 aa, synthetic construct
MALPVTALLLPLALLLHAARPLPVLTQPPSASGTPGQRVTISCSGSSSNIGSNAVDWYQQLPGTAPKLLIYTNNRRPSGVPDRFSGSKSGTSGSLAISGLQFEDEADYYCAAWDDSLSGVIFGGGTKVTVLGGGGSGGGGSGGGGSQVQLVQSGAEVKKPGSSVKVSCKASEDTFSSYVISWVRQAPGQGLEWMGAFIPLLGRVHYAQEFQGRLTITADESTGTAYMELKGLRSDDTAMYYCARDQGFAGDDAFDIWGQGTMVTVSS

SEQ ID NO: 175 - protein, 266 aa, synthetic construct
MALPVTALLLPLALLLHAARPQPVLTQDPVVSVALGQTVRITCQGDSLRSYYATWYQQKPGLAPVRVIFGKNSRPSGIPDRFSGSSSGNTASLTITGAQAEDEADYYCNSWDNSANHPVFGGGTKLTVLGGGGSGGGGSGGGGSEVQLVQSGAEVRKPGSSVKVSCKASGDNFNNYGITWVRQAPGQGLEWMGRLITVLGLDNYAQKFQGRLTITADESTGTAYMELTGLEPEDTAVYYCARDIMSPEAIDAFDVWGQGTLVTVSS

SEQ ID NO: 176 - protein, 265 aa, synthetic construct
MALPVTALLLPLALLLHAARPDIQMTQSPSSLSASVGDRVTITCRASQDVSNYLNWYQQKPGKAPKLLIYGASNRQTGVPSRFSGSGSGTDFTLTISSLQPEDFATYYCQQEDRFPTTFGQGTKVEIKGGGGSGGGGSGGGGSEVQLVQSGAEVRKPGSSVKVSCKASGDNFNNYGITWVRQAPGQGLEWMGRLITVLGLDNYAQKFQGRLTITADESTGTAYMELTGLEPEDTAVYYCARDIMSPEAIDAFDVWGQGTLVTVSS

SEQ ID NO: 177 - protein, 270 aa, synthetic construct
MALPVTALLLPLALLLHAARPEIVLTQSASSLSASVGDRVTITCRTSQNIYRYLNWYQQKPGKAPKLLIFGASTLQTGVPSRFSGSGSGTDFTLTITGLQPEDLATYYCQHSFKLPFTFGQGTRLEIKGGGGSGGGGSGGGGSQMLVQSGAEVKRPGSSVTVSCETSGDTFSRYAISWLRQAPGQGLEWLGWISGNSGRADYAQNFQGRVTMTTDRSTSTAYLNLRSLRSDDTAVYYCARDAPMLFGVRGGGYYFGMDVWGQGTTVTVSS

SEQ ID NO: 178 - protein, 258 aa, synthetic construct
MALPVTALLLPLALLLHAARPQPVLTQDPAVSVALGQTVRITCQGDSLRKYYASWYQQKPGQAPRLVMYDENDRPSGIPDRFSGSSSGNTASLTITGAQADDEADYYCNSRDSSGHHVVFGGGTKVTVLGGGGSGGGGSGGGGSQMQLVQSGAEVRRPGSSVKVSCKTSGGTLNNYDINWVRQAPGQGLEWMGWMNPTSGQADFAQKFQGRVSMTRDTSINTAFMELNTLRSEDTAVYYCVRRGGKYWGQGTLVTVSS

SEQ ID NO: 179 - protein, 267 aa, synthetic construct
MALPVTALLLPLALLLHAARPSYELTQPPSVSVAPGQTARITCGGDALPNNSVRWYQQKPGQAPVLVIYGNSERPSGIPERFSGSNSGNTATLTISGTQAEDEADYYCQVSDKSTKYSVFGGGTKLTVLGGGGSGGGGSGGGGSEVQLVESGGGLVQPGGSLRLSCAASGFTFSNYWMSWVRQAPGKGLEWVAGISASGGNKYYADSVKGRFTISRDNSKNTLYLQMNSLRAEDTAVYYCAKGPIQLKRHKYVLDYWGQGTLVTVSS

SEQ ID NO: 180 - protein, 108 aa, synthetic construct; identical to SEQ ID NO: 86

SEQ ID NO: 181 - protein, 122 aa, synthetic construct; identical to SEQ ID NO: 87

SEQ ID NO: 182 - protein, 127 aa, synthetic construct
QMLVQSGAEVKRPGSSVTVSCETSGDTFSRYAISWLRQAPGQGLEWLGWISGNSGRADYAQNFQGRVTMTTDRSTSTAYLNLRSLRSDDTAVYYCARDAPMLFGVRGGGYYFGMDVWGQGTTVTVSS

SEQ ID NO: 183 - protein, 107 aa, synthetic construct
EIVLTQSASSLSASVGDRVTITCRTSQNIYRYLNWYQQKPGKAPKLLIFGASTLQTGVPSRFSGSGSGTDFTLTITGLQPEDLATYYCQHSFKLPFTFGQGTRLEIK

SEQ ID NO: 184 - protein, 114 aa, synthetic construct
QMQLVQSGAEVRRPGSSVKVSCKTSGGTLNNYDINWVRQAPGQGLEWMGWMNPTSGQADFAQKFQGRVSMTRDTSINTAFMELNTLRSEDTAVYYCVRRGGKYWGQGTLVTVSS

SEQ ID NO: 185 - protein, 108 aa, synthetic construct
QPVLTQDPAVSVALGQTVRITCQGDSLRKYYASWYQQKPGQAPRLVMYDENDRPSGIPDRFSGSSSGNTASLTITGAQADDEADYYCNSRDSSGHHVVFGGGTKVTVL

SEQ ID NO: 186 - protein, 123 aa, synthetic construct
EVQLVESGGGLVQPGGSLRLSCAASGFTFSNYWMSWVRQAPGKGLEWVAGISASGGNKYYADSVKGRFTISRDNSKNTLYLQMNSLRAEDTAVYYCAKGPIQLKRHKYVLDYWGQGTLVTVSS

SEQ ID NO: 187 - protein, 108 aa, synthetic construct
SYELTQPPSVSVAPGQTARITCGGDALPNNSVRWYQQKPGQAPVLVIYGNSERPSGIPERFSGSNSGNTATLTISGTQAEDEADYYCQVSDKSTKYSVFGGGTKLTVL

SEQ ID NO: 188 - protein, 20 aa, synthetic construct
GGGGSGGGGSGGGGSGGGGS

SEQ ID NO: 189 - DNA, 19 bp, synthetic construct
  source 1..19: mol_type = other DNA
tgcatttgaactccttgcc

SEQ ID NO: 190 - DNA, 20 bp, synthetic construct
  source 1..20: mol_type = other DNA
ccatcaatcttccacttgac

SEQ ID NO: 191 - protein, 10 aa, synthetic construct
APFTKPSPFT

SEQ ID NO: 192 - protein, 9 aa, synthetic construct
DIRLSGRTD

SEQ ID NO: 193 - protein, 10 aa, synthetic construct
GSVFRFYGMG

SEQ ID NO: 194 - protein, 16 aa, synthetic construct
QRNIPRIGLGSRNYDY

SEQ ID NO: 195 - protein, 10 aa, synthetic construct
AISGGGIYTD

SEQ ID NO: 196 - protein, 10 aa, synthetic construct
GRTASSNAMG

SEQ ID NO: 197 - protein, 15 aa, synthetic construct
LSTNYPFLRPRDANY

SEQ ID NO: 198 - protein, 10 aa, synthetic construct
TISRDGTRTY

SEQ ID NO: 199 - protein, 10 aa, synthetic construct
GRTFTTYRMG

SEQ ID NO: 200 - protein, 123 aa, synthetic construct
DVQLQESGGGLVQAGGSLRLSCAASGRTFSTYRITWFRQAPGKQREFVATISRSSSNPYYADSVKGRFTISRDNAKNTVYLQMNSLRPEDTAVYSCAGSTAYPFLSPRDANYWGQGTQVTVSS

SEQ ID NO: 201 - protein, 114 aa, synthetic construct
DVQLQESGGGSVQPGGSLRLSCVASGSISSDNAMGWYRQTAPWKQRESVASLTRDGSAIYADSVKGRFTISRDNAANTVYLQMNSLKPEDTAVYYCFRTPPTYWGQGTQVTVSS

SEQ ID NO: 202 - protein, 113 aa, synthetic construct
DVQLQESGGGSVQPGGSLRLSCVVSGSGSSINSMGWYRQAPGKQRESVASITRAGSAIYADSVKGRFTISRDNAANTAYLQMNSLKPEDTAVYYCFHTPPTYWGQGTQVTVSS

SEQ ID NO: 203 - protein, 117 aa, synthetic construct
DVQLQESGGGLVQPGGSLRLSCSASGSGFRFYGMGWYRQAPGKQRELVADIRLSGRAEYADSVKGRFTISRDNAKNTVYLQMNSLKPEDTAVYYCNAPFTSPSPFAWGQGTQVTVSS

SEQ ID NO: 204 - protein, 119 aa, synthetic construct
DVQLQESGGGLVQAGGSLRLSCAASGRTFSNLAMGWFRQAPGKAREFVAAISGSGGSIFYADSVKGRFTISRDNAKNTMYLQMNSLKPEDTAVYYCAAGPYGSPDFDSWGQGTQVTVSS

SEQ ID NO: 205 - protein, 123 aa, synthetic construct
DVQLQESGGGSVQAGGSLRLSCAASGRTFTTYRIAWFRQAPGKEREFVATISRDGTRTYYADSVKGRFTISRDNAKNTVYLQMNSLRPEDTAVYYCAGSTDYPFLRPRDANYWGQGTQVTVSS

SEQ ID NO: 206 - protein, 123 aa, synthetic construct
DVQLQESGGGSVQAGGSLRLSCAASGRTFTTYRMGWFRQAPGKQREFVATISRDGTRTYYADSVKGRFTISRDNAENTVYLQMNSLRPEDTAAYYCAVSTDYPFLRPRDAAYWGQGTQVTVSS

SEQ ID NO: 207 - protein, 126 aa, synthetic construct
DVQLQESGGGLVQAGESLRLSCAASGRTSSTYTMAWFRQAPGKDREFVGRIRWNSGSADYAGSVKGRFTISRDNAKNTVYLQMNSLKPEDTAVYYCAAASWGYSSSYYSSRGYDVWGQGTQVTVSS

SEQ ID NO: 208 - protein, 124 aa, synthetic construct
DVQLQESGGGLVQAGGSLRLACAASGRTASSNAMGWFRQAPGKEREFVAAISGGGIYTDYADSVKGRFTISRDNSKNTMYLQMNSLKPEDTAVYYCAQRNIPRIGLGSGNYDYWGQGTQVTVSS

SEQ ID NO: 209 - protein, 116 aa, synthetic construct
DVQLQESGGGLVQVGGSLTLSCAASGRTFSKYTMSWFRQAPGQEREFVATITSSGRSTSYGDSVKGRFTISRDNAKNTMYLQMNSLKFEDTAVYYCRAADSVSAYWGQGTQVTVSS

SEQ ID NO: 210 - protein, 113 aa, synthetic construct
DVQLQESGGGSVQPGGSLRLSCVASGSGSSIAAMGWYRQVPGKQRESVASLTREGRVIYADSVKGRFTISRDNAANTVYLQMNSLKPEDTAVYYCFRTPPTLWGQGTQVTVSS

SEQ ID NO: 211 - protein, 113 aa, synthetic construct
DVQLQESGGGSVQPGGSLRLSCVASGNISSNNAMGWYRQAPGKQRESVASMPRGGTAIYADSVKGRFTISRDNAANTVYLQMNSLKPEDTAVYYCFRTPPTYWGQGTQVTVSS

SEQ ID NO: 212 - protein, 123 aa, synthetic construct
DVQLQESGGGLVQAGGSLRLSCAASGRTFSTHRITWFRQAPGKQREFVATISRSSSNPYYADSVKGRFTISRDNAKNTVYLQMNSLRPEDTAVYSCAGSTAYPFLSPRDANYWGQGTQVTVSS

SEQ ID NO: 213 - protein, 123 aa, synthetic construct
DVQLQESGGGLVQTGGSLRLSCAASGRFFSTYAMGWFRQAPGKEREFVAAITRGGAGTLYADSAKGRFTISRDNAKNTVYLQMNSLKAEDTGVYYCAPNNGSRSWYGYEYDYWGQGTQVTVSS

SEQ ID NO: 214 - protein, 117 aa, synthetic construct
DVQLQESGGGLVQPGGSLRLECSAPGNIFRFYAMGWYRQAPGKQRELVADIRLSGSTNIANSVKDRFTISRDNAKNTVYLQMNSLKPEDTAVYYCNAPFTKPSPFAWGQGTQVTVSS

SEQ ID NO: 215 - protein, 124 aa, synthetic construct
DVQLQESGGGLVQAGGSLRLSCTASGLTFRSYAMGWFRQAPGKEREFLAHISVSGSITPYADSVKGRFTLSRDNAKNTVYLQMNSLKPEDTAVYYCAARRVGSWYSTKEEYDYWGQGTQVTVSS

SEQ ID NO: 216 - protein, 117 aa, synthetic construct
DVQLQESGGGLVQPGGSLRLSCSASGNVFRFYGMGWYRQAPGKQRELVADIRLSGRTDYADSVKGRFTISRDNAGNTVYLQMNGLKPEDTAVYYCNAPFTKPSPFTWGQGTQVTVSS

SEQ ID NO: 217 - protein, 118 aa, synthetic construct
DVQLQESGGGLVQPGGSLRLSCAASGFTFGSYAMGWYRQAPGNERELVALISVAGGITHYADSVKGRFRISRDNAENMVYLQMNSLKPEDTAVYYCSARRERFTTAYWGQGTQVTVSS

SEQ ID NO: 218 - protein, 117 aa, synthetic construct
DVQLQESGGGLVQPGGSLRLSCSASGSVFRFYGMGWYRQAPGKQRELVADIRLSGRTDYADPVKGRFTISRDNAGNTVYLQMNGLKPEDAAVYYCNAPFTKPSPFTWGQGTQVTVSS

SEQ ID NO: 219 - protein, 123 aa, synthetic construct
DVQLQESGGGSVQAGGSLRLSCAASGRTFTTYRMGWFRQAPGKQREFVATISRDGTRTYYADSVKGRFTISRDNAESTVYLQMNSLRPEDTAVYYCAVSTDYPFLRPRDAAYWGQGTQVTVSS

SEQ ID NO: 220 - protein, 119 aa, synthetic construct
DVQLQESGGGLVQPGGSLRLSCAASGFTFSNYAMTWVRQAPGKGAEWVSAINIYGDITHYADSVKGRFTISRDNAKNTLYLQMNSLQLDDTAVYYCGTKLDGNSNKVYRGQGTQVTVSS

SEQ ID NO: 221 - protein, 125 aa, synthetic construct
DVQLQESGGGLVQAGGSLSLSCAASGQTFSRNVMGWFRQAPGKEREFVAAITVGGAGTYYADSVKGRFTISRDNPKNTVYLQMNSLKPEDTAVYYCAANRRFARPVVAIASYDVWGQGTQVTVSS

SEQ ID NO: 222 - protein, 123 aa, synthetic construct
DVQLQESGGGSVQAGDSLRLSCAASGRTFTTYRMAWFRQAPGKQREFVATVSRDGTRTYYADSVKGRFTISRDNAKNTVYLQMSSLRPEDTAAYYCAVSTDYPFLRPRDAAYWGQGTQVTVSS

SEQ ID NO: 223 - protein, 123 aa, synthetic construct
DVQLQESGGGSVQAGGSLRLSCAASGRTFTTYRMSWFRQAPGNEREFVATISPDGTRTYYADSVKGRFTISRDNAKNTAYLQMNSLRPEDTAVYYCAGSTVYPFLRPRDANYWGQGTQVTVSS

SEQ ID NO: 224 - protein, 123 aa, synthetic construct
DVQLQESGGGSVQAGDTLRLSCAASGRTFATYRMAWFRQAPGKQREFVATVSRDGTSTYYADSVKGRFTISRDNAKNTVYLQMNSLRPEDTAVYYCAVSTDYPFLRPRDAAYWGQGTQVTVSS

SEQ ID NO: 225 - protein, 140 aa, synthetic construct
DVQLQESGGGLVQAGGSLRLACAASGRTASSNAMGWFRQAPGKEREFVAAISGGGIYTDYADSVKGRFTISRDNSKNTMYLQMNSLKPEDTAVYYCAQRNIPRIGSGSRNYDYWGQGTQVTVSSQRNIPRIGSGSRNYDY

SEQ ID NO: 226 - protein, 123 aa, synthetic construct
DVQLQESGGGSVQAGGSLRLSCAASGRTFGTYRMTWFRQAPGKQREFVATISRNGGSTYYADSVKGRFTISRDNAKNTVFLQMNSLRPEDTAVYYCAGSTAYPFLSPRDANYWGQGTQVTVSS

SEQ ID NO: 227 - protein, 123 aa, synthetic construct
DVQLQESGGGSVQAGGSLRLSCAASGRTFTTYRMSWFRQAPGNEREFVATISPDGTRTYYADSVKGRFTISRDNAKNTVYLQMNSLRPEDTAVYYCAGSTVYPFLRPRDANYWGQGTQVTVSS

SEQ ID NO: 228 - protein, 123 aa, synthetic construct
DVQLQESGGGSVQAGGSLRLSCAASGRTFTTYRMAWFRQAPGKQREFVATISRNGGSTYYADSVKGRFTISRDNAKNTVFLQMNSLRPEDTAVYYCAGSTDYPFLRPRDANYWGQGTQVTVSS

SEQ ID NO: 229 - protein, 117 aa, synthetic construct
DVQLQESGGGLVQPGGSLRLECSAPGSIFRFYAMGWYRQAPGKQRELVADIRLSGRAVYADSVKGRFTISRDNAKNTVYLQMDSLKPEDTAVYYCNAPFTKPSPFAWGQGTQVTVSS

SEQ ID NO: 230 - protein, 123 aa, synthetic construct
DVQLQESGGGSVQAGGSLRLSCAASERTFTTYRMAWFRQAPGKQREFVATISRDGTRTYYADSVKGRFTISSDNAKNTVYLQMNSLRPEDTAVYYCAGSTDYPFLRPRDANYWGQGTQVTVSS

SEQ ID NO: 231 - protein, 123 aa, synthetic construct
DVQLQESGGGSVQAGGSPRLSCAASGRTFTTYRIAWFRQAPGKEREFVATISRDGTRTYYADSVKGRFTISRDNARNTVYLQMNSLRPEDTAVYYCAGSTDYPFLRPRDANYWGQGTQVTVSS

SEQ ID NO: 232 - protein, 113 aa, synthetic construct
DVQLQESGGGSVQPGGSLRLSCVASGSGSSIVSMGWYRQTPGKQRESVASITSDGSRLYADSVKGRFTISRVNAANTVYLQMNSLKPEDTATYYCFLTPPTYWGQGTQVTVSS

SEQ ID NO: 233 - protein, 123 aa, synthetic construct
DVQLQESGGGSVQAGGSLRLSCAASGRTFTTYRIAWFRQAPGKEREFVATISRDGTRTYYADSVKGRFTTSRDNAKNTVYLQMNSLRPEDTAVYYCAGSTDYPFLRPRDANYWGQGTQVTVSS

SEQ ID NO: 234 - protein, 123 aa, synthetic construct
DVQLQESGGGSVQAGGSLRLSCAASGRTFTTYRMGWFRQAPGKEREFVATISRDGTRPYYADSVKGRFTISRDNTKNTVYLQMNSLRPEDTAVYYCARSTDYPFLRPRDANYWGQGTQVTVSS

SEQ ID NO: 235 - protein, 123 aa, synthetic construct
DVQLQESGGGSVQAGGSLRLSCAASGRTFTTYRMSWFRQAPGNEREFVATISPDGTRTYYADSVKGRFTISRDNAKNTVYLHMNSLRPEDTAVYYCAGSTVYPFLRPRDANYWGQGTQVTVSS

SEQ ID NO: 236 - protein, 123 aa, synthetic construct
DVQLQESGGGSVQAGGSLRLSCAASGRTFMTYRMGWFRQAPGNEREFVGNISPDGTRTYYADSVKGRFTISRDNAKNTVYLQMNSLRPEDTAVYYCAGSTVYPFLRPRDANYWGQGTQVTVSS

SEQ ID NO: 237 - protein, 121 aa, synthetic construct
DVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSS

SEQ ID NO: 238 - protein, 117 aa, synthetic construct
DVQLQESGGGLVQPGGSLRLSCSASGSVFRFYGMGWYRQAPGKQRELVADIRLSGRTNYADSVKGRFTISRDNAGNTVYLQMNSLKPEDTAVYYCNAPFTIPSPFAWGQGTQVTVSS

SEQ ID NO: 239 - protein, 114 aa, synthetic construct
DVQLQESGGGSVQPGGSLRLSCVASGSISSDNAMGWYRQAAPWKQRESVASLTRDGSAIYADSVKGRFTISRDNAANTVYLQMNSLKPEDTAVYYCFRTPPTYWGQGTQVTVSS

SEQ ID NO: 240 - protein, 123 aa, synthetic construct
DVQLQESGGGSVQAGGSLRLSCAASGRTFSTYRMTWFRQAPGKQREFVATISRNGGSTYYADSVKGRFTISRDNAKNTVFLQMNSLRPEDTAVYYCAGSTAYPFLSPRDANYWGQGTQVTVSS

SEQ ID NO: 241 - protein, 123 aa, synthetic construct; identical to SEQ ID NO: 200

SEQ ID NO: 242 - protein, 117 aa, synthetic construct
DVQLQESGGGLVQPGGSLRLSCSANGSVFRFYGMGWYRQAPGKQRELVADIRLSGRADYADSVKGRFTISRDNAGNTVYLQMNSLKPEDTAVYYCNAPFTIPSPFAWGQGTQVTVSS

SEQ ID NO: 243 - protein, 114 aa, synthetic construct
DVQLQESGGGSVQPGGSLRLSCVASGSGSSINTSMGWYRQRPGKQRESVASMPRGGGTIYADSVKGRFTISRDNAANTVYLQMNNLKPEDTAVYYCFRTPPTYWGQGTQVTVSS

SEQ ID NO: 244 - protein, 113 aa, synthetic construct
DVQLQESGGGSVQPGGSLRLSCVASGSISSINAMGWYRQAPGKQHESVASMPRSGTAIYADSVKGRFTISRDNAANTVYLQMNSLKPEDTAKYYCFHTPPTYWGQGTQVTVSS

SEQ ID NO: 245 - protein, 114 aa, synthetic construct
DVQLQESGGGSVQPGGSLRLSCVASGSGSSINTSMGWYRQTPGKQRESVASMPRGGGAIYADSVKGRFTISRDYAANTVYLQMNSLKPEDTAVYYCFQTPPTYWGQGTQVTVSS

SEQ ID NO: 246 - protein, 117 aa, synthetic construct
DVQLQESGGGLVQPGGSLRLSCSASGSIFRFYGMGWYRQTPGKQRELVAYIRLSGRTDYADSVKGRFTISRDNAGNTVYLQMNSLKPEDTAVYYCNPPFTKPSPFAWGQGTPVTVSS

SEQ ID NO: 247 - protein, 123 aa, synthetic construct
DVQLQESGGGSVQAGGSLRLSCAASGRTFSTYRMAWFRQAPGKQREFVATISPNGASTYYADSVKGRFTISRDNAKNTLYLQMNSLRPEATAVYYCAGSTDYPFLRPRDANFWGQGTQVTVSS

SEQ ID NO: 248 - protein, 117 aa, synthetic construct
DVQLQESGGGLVQPGGSLRLSCSASGSIFRFYGMGWYRQAPGKQRELVADIRLSGRTDYADSVKGRFTISRDNAGNTVYLQMNSLKPEDTAVYYCNAPFTKPSPFAWGQGTQVTVSS

SEQ ID NO: 249 - protein, 123 aa, synthetic construct
DVQLQESGGGSVQAGGSLRLSCAASGRTFTTYRMSWFRQAPGNERGFVATISPDGTRTYYADSVKGRFTISRDNAKNTVYLQMNSLRPEDTAVYYCAGSTVYPFLRPRDANYWGQGTQVTVSS

SEQ ID NO: 250 - protein, 123 aa, synthetic construct
DVQLQESGGGSVQAGGSLRLSCAASGRTFSTYRMGWFRQAPGKQREFVAEISRNGGYTYYADSVKGRFTISRDNAKNTVYLQMNSLRPEDTAVYYCAGSTTYPFLSPRDANYWGQGTQVTVSS

SEQ ID NO: 251 - protein, 123 aa, synthetic construct
DVQLQESGGGSVQAGGSLRLSCAASGRTFTTYRMGWFRQAPGKQREFVATISRDGTRTYYADSVKGRFTISRDNAENTVYLQMNSLRPEDTAVYYCAVSTDYPFLRPRDAAYWGQGTQVTVSS

SEQ ID NO: 252 - protein, 114 aa, synthetic construct
DVQLQESGGGSVQPGGSLRLSCVASGSGSSINTSMGWYRQAPGKQRESVASMPRGGGALYADSVKGRFTISRDNAANTVYLQMNSLKPEDTAVYYCFQTPPTFWGQGTQVTVSS

SEQ ID NO: 253 - protein, 123 aa, synthetic construct
DVQLQESGGGSVQAGGSLRLSCAASGRTFTTDRMAWFRQAPGKEREFVATISRDGTRPYYADSVKGRFTISRDNAKNTVYLQMNSLRPEDTAVYYCAGSTVYPFLRPRDANYWGQGTQVTVSS

SEQ ID NO: 254 - protein, 123 aa, synthetic construct
DVQLQESGGGSVQAGGSLRLSCAASEGTFSTYRMTWFRQAPGKQREFVATISRNGGSTYYVDSVKGRFTISRDNAKNTVFLQMNSLRPEDTAVYYCAGSTAYPFLSPRDANYWGQGTQVTVSS

SEQ ID NO: 255 - protein, 123 aa, synthetic construct
DVQLQESGGGSVQAGGSLRLSCAASGRTFTTYRVAWFRQAPGKEREFVATVSRDGTRAYYADSVKGRFTISRDNAKNTVYLQMNSLRPEDTAVYYCAGSTDYPFLRPRDANYWGQGTQVTVSS

SEQ ID NO: 256 - protein, 123 aa, synthetic construct
DVQLQESGGGSVQAGGSLRLSCAASGRTFTTYRMSWFRQAPGNEREFVATISPDGTRTYYADSVKGRFTISRDNAKNTVYLQMNSLRPEDTAVYYCAGSTVYPFLRPRDANYWGPGTQVTVSS

SEQ ID NO: 257 - protein, 123 aa, synthetic construct
DVQLQESGGGSVQAGGSLRLSCVASGGTVTTYRMAWFRQAPGKQREFVATISRDGTSTYYADSVKGRFTISRDNAKNTAYLQMNSLRPEDTAVYYCAGSTTYPFLRPRDANYWGQGTQVTVSS

SEQ ID NO: 258 - protein, 119 aa, synthetic construct
DVQLQESGGGLVQPGDSLRLSCAAGGSSLNIYAIGWFRQAPGKEREGVQCISRSDGITFYDDSVKGRFTVSRDRAKGTVYLQMNGLQPEDAAVCYCAAGYRCALYMDYWGKGTQVTVSS

SEQ ID NO: 259 - protein, 123 aa, synthetic construct
DVQLQESGGGSVQAGDSLRLSCAASGRTFTTYRVAWFRQAPGKQREFVATVSRDGTRTYYADSVKGRFTISRDNAKNTVYLQMNSLRPEDTAVYYCAVSTDYPFLRPRDAAYWGQGTQVTVSS

SEQ ID NO: 260 - protein, 113 aa, synthetic construct
DVQLQESGGGSVQPGGSLRLSCVASGSISSNVSMGWYRQAPGKQRESVASMPRGGSAIYADSVKGRFVISRDNAANTVYLQMNSLKPEDTAVYYCFRTPPTYWGQGTQVTVSS

SEQ ID NO: 261 - protein, 117 aa, synthetic construct
DVQLQESGGGLVQPGGSLRLECSAPGNIFRFYAMGWYRQAPGKQRELVADIRLSGSTNIANSVKGRFTISRDNAKNTVYLQMNSLKPEDTAVYYCNAPFTKPSPFAWGQGTQVTVSS

SEQ ID NO: 262 - protein, 123 aa, synthetic construct
DVQLQESGGGSVQAGGSLRLSCAASGRTFTTYRMGWLRQAPGKQREFVATISRDGTRTYYADSVEGRFTISRDNAKNTAYLQMNSLRPEDTAVYYCALSTNYPFLRPRDANYWGQGTQVTVSS

SEQ ID NO: 263 - protein, 114 aa, synthetic construct
DVQLQESGGGSVQPGGSLRLSCVASGSGSSINTSMGWYRQAPGKQRESVASMPRGGGAIYADSVKGRFTISRDNAANTVYLQMNSLKPEDTAVYYCFQTPPTYWGQGTQVTVSS

SEQ ID NO: 264 - protein, 123 aa, synthetic construct
DVQLQESGGGSVQAGGSLRLSCAASGRTFTTYRMGWFRQAPGKEREFVATISGDGTRPYYADSVKGRFTISRDNTKNTVYLQMNSLRPEDTAVYYCAGSTDYPFLRPRDANYWGQGTQVTVSS

SEQ ID NO: 265 - protein, 123 aa, synthetic construct; identical to SEQ ID NO: 227

SEQ ID NO: 266 - protein, 123 aa, synthetic construct
DVQLQESGGGSVQAGGSLRLSCAASEGTFSTYRMTWFRQAPGKQREFVATISRNGGSTYYADSVKGRFTISRDNAKNTVFLQMNSLRPEDTAVYYCAGSTAYPFLSPRDANYWGQGTQVTVSS

SEQ ID NO: 267 - protein, 123 aa, synthetic construct
DVQLQESGGGSVQAGGSLRLSCAASGRTFTTYRMGWFRQAPGKQREFVATISRDGTRTYYADSVEGRFTISRDNAKNTVYLQMNSLRPEDTAVYYCALSTNYPFLRPRDANYWGQGTQVTVSS

SEQ ID NO: 268 - protein, 124 aa, synthetic construct
DVQLQESGGGLVQAGGSLRLACAASGRTASSNAMGWFRQAPGKEREFVAAISGGGIYTDYADSVKGRFTISRDNSKNTMYLQMNSLKPEDTAVYYCAQRNIPRIGLGSRNYDYWGQGTQVTVSS

SEQ ID NO: 269 - protein, 117 aa, synthetic construct
DVQLQESGGGLVQPGGSLRLSCSASGSVFRFYGMGWYRQAPGKQRELVADIRLSGRTDYADSVKGRFTISRDNAGNTVYLQMNGLKPEDTAVYYCNAPFTKPSPFTWGQGTQVTVSS

SEQ ID NO: 270 - protein, 10 aa, synthetic construct
GRTFSTYRIT

SEQ ID NO: 271 - protein, 10 aa, synthetic construct
TISRSSSNPY

SEQ ID NO: 272 - protein, 15 aa, synthetic construct
GSTAYPFLSPRDANY

SEQ ID NO: 273 - protein, 10 aa, synthetic construct
GSISSDNAMG

SEQ ID NO: 274 - protein, 9 aa, synthetic construct
SLTRDGSAI

SEQ ID NO: 275 - protein, 6 aa, synthetic construct
RTPPTY

SEQ ID NO: 276 - protein, 10 aa, synthetic construct
GSGSSINSMG

SEQ ID NO: 277 - protein, 9 aa, synthetic construct
SITRAGSAI

SEQ ID NO: 278 - protein, 6 aa, synthetic construct
HTPPTY

SEQ ID NO: 279 - protein, 10 aa, synthetic construct
GSGFRFYGMG

SEQ ID NO: 280 - protein, 9 aa, synthetic construct
DIRLSGRAE

SEQ ID NO: 281 - protein, 10 aa, synthetic construct
APFTSPSPFA

SEQ ID NO: 282 - protein, 10 aa, synthetic construct
GRTFSNLAMG

SEQ ID NO: 283 - protein, 10 aa, synthetic construct
AISGSGGSIF

SEQ ID NO: 284 - protein, 11 aa, synthetic construct
AGPYGSPDFDS

SEQ ID NO: 285 - protein, 10 aa, synthetic construct
GRTFTTYRIA

SEQ ID NO: 286 - protein, 10 aa, synthetic construct
TISRDGTRTY

SEQ ID NO: 287 - protein, 15 aa, synthetic construct
GSTDYPFLRPRDANY

SEQ ID NO: 288 - protein, 10 aa, synthetic construct
GRTFTTYRMG

SEQ ID NO: 289 - protein, 10 aa, synthetic construct
TISRDGTRTY

SEQ ID NO: 290 - protein, 15 aa, synthetic construct
VSTDYPFLRPRDAAY

SEQ ID NO: 291 - protein, 10 aa, synthetic construct
GRTSSTYTMA

SEQ ID NO: 292 - protein, 10 aa, synthetic construct
RIRWNSGSAD

SEQ ID NO: 293 - protein, 18 aa, synthetic construct
AASWGYSSSYYSSRGYDV

SEQ ID NO: 294 - protein, 10 aa, synthetic construct
GRTASSNAMG

SEQ ID NO: 295 - protein, 10 aa, synthetic construct
AISGGGIYTD

SEQ ID NO: 296 - protein, 16 aa, synthetic construct
QRNIPRIGLGSGNYDY

SEQ ID NO: 297 - protein, 10 aa, synthetic construct
GRTFSKYTMS

SEQ ID NO: 298 - protein, 10 aa, synthetic construct
TITSSGRSTS

SEQ ID NO: 299 - protein, 8 aa, synthetic construct
AADSVSAY

SEQ ID NO: 300 - protein, 10 aa, synthetic construct
GSGSSIAAMG

SEQ ID NO: 301 - protein, 9 aa, synthetic construct
SLTREGRVI

SEQ ID NO: 302 - protein, 6 aa, synthetic construct
RTPPTL

SEQ ID NO: 303 - protein, 10 aa, synthetic construct
GNISSNNAMG

SEQ ID NO: 304 - protein, 9 aa, synthetic construct
SMPRGGTAI

SEQ ID NO: 305 - protein, 6 aa, synthetic construct
RTPPTY

SEQ ID NO: 306 - protein, 10 aa, synthetic construct
GRTFSTHRIT

SEQ ID NO: 307 - protein, 10 aa, synthetic construct
TISRSSSNPY

SEQ ID NO: 308 - protein, 15 aa, synthetic construct
GSTAYPFLSPRDANY

SEQ ID NO: 309 - protein, 10 aa, synthetic construct
GRFFSTYAMG

SEQ ID NO: 310 - protein, 10 aa, synthetic construct
AITRGGAGTL

SEQ ID NO: 311 - protein, 15 aa, synthetic construct
PNNGSRSWYGYEYDY

SEQ ID NO: 312 - protein, 10 aa, synthetic construct
GNIFRFYAMG

SEQ ID NO: 313 - protein, 9 aa, synthetic construct
DIRLSGSTN

SEQ ID NO: 314 - protein, 10 aa, synthetic construct
APFTKPSPFA

SEQ ID NO: 315 - protein, 10 aa, synthetic construct
GLTFRSYAMG

SEQ ID NO: 316 - protein, 10 aa, synthetic construct
HISVSGSITP

SEQ ID NO: 317 - protein, 16 aa, synthetic construct
ARRVGSWYSTKEEYDY

SEQ ID NO: 318 - protein, 10 aa, synthetic construct
GNVFRFYGMG

SEQ ID NO: 319 - protein, 9 aa, synthetic construct
DIRLSGRTD

SEQ ID NO: 320 - protein, 10 aa, synthetic construct
APFTKPSPFT

SEQ ID NO: 321 - protein, 10 aa, synthetic construct
GFTFGSYAMG

SEQ ID NO: 322 - protein, 10 aa, synthetic construct
LISVAGGITH

SEQ ID NO: 323 - protein, 10 aa, synthetic construct
ARRERFTTAY

SEQ ID NO: 324 - protein, 10 aa, synthetic construct
GSVFRFYGMG

SEQ ID NO: 325 - protein, 9 aa, synthetic construct
DIRLSGRTD

SEQ ID NO: 326 - protein, 10 aa, synthetic construct
APFTKPSPFT

SEQ ID NO: 327 - protein, 10 aa, synthetic construct
GRTFTTYRMG

SEQ ID NO: 328 - protein, 10 aa, synthetic construct
TISRDGTRTY

SEQ ID NO: 329 - protein, 15 aa, synthetic construct
VSTDYPFLRPRDAAY

SEQ ID NO: 330 - protein, 10 aa, synthetic construct
GFTFSNYAMT

SEQ ID NO: 331 - protein, 10 aa, synthetic construct
AINIYGDITH

SEQ ID NO: 332 - protein, 11 aa, synthetic construct
TKLDGNSNKVY

SEQ ID NO: 333 - protein, 10 aa, synthetic construct
GQTFSRNVMG

SEQ ID NO: 334 - protein, 10 aa, synthetic construct
AITVGGAGTY

SEQ ID NO: 335 - protein, 17 aa, synthetic construct
ANRRFARPVVAIASYDV

SEQ ID NO: 336 - protein, 10 aa, synthetic construct
GRTFTTYRMA

SEQ ID NO: 337 - protein, 10 aa, synthetic construct
TVSRDGTRTY

SEQ ID NO: 338 - protein, 15 aa, synthetic construct
VSTDYPFLRPRDAAY

SEQ ID NO: 339 - protein, 10 aa, synthetic construct
GRTFTTYRMS

SEQ ID NO: 340 - protein, 10 aa, synthetic construct
TISPDGTRTY

SEQ ID NO: 341 - protein, 15 aa, synthetic construct
GSTVYPFLRPRDANY

SEQ ID NO: 342 - protein, 10 aa, synthetic construct
GRTFATYRMA

SEQ ID NO: 343 - protein, 9 aa, synthetic construct
TVSRDGTST

SEQ ID NO: 344 - protein, 15 aa, synthetic construct
VSTDYPFLRPRDAAY

SEQ ID NO: 345 - protein, 10 aa, synthetic construct
GRTASSNAMG

SEQ ID NO: 346 - protein, 10 aa, synthetic construct
AISGGGIYTD

SEQ ID NO: 347 - protein, 16 aa, synthetic construct
QRNIPRIGSGSRNYDY

SEQ ID NO: 348 - protein, 10 aa, synthetic construct
GRTFGTYRMT

SEQ ID NO: 349 - protein, 10 aa, synthetic construct
TISRNGGSTY

SEQ ID NO: 350 - protein, 15 aa, synthetic construct
GSTAYPFLSPRDANY

SEQ ID NO: 351 - protein, 10 aa, synthetic construct
GRTFTTYRMS

SEQ ID NO: 352 - protein, 10 aa, synthetic construct
TISPDGTRTY

SEQ ID NO: 353 - protein, 15 aa, synthetic construct
GSTVYPFLRPRDANY

SEQ ID NO: 354 - protein, 10 aa, synthetic construct
GRTFTTYRMA

SEQ ID NO: 355 - protein, 10 aa, synthetic construct
TISRNGGSTY

SEQ ID NO: 356 - protein, 15 aa, synthetic construct
GSTDYPFLRPRDANY

SEQ ID NO: 357 - protein, 10 aa, synthetic construct
GSIFRFYAMG

SEQ ID NO: 358 - protein, 9 aa, synthetic construct
DIRLSGRAV

SEQ ID NO: 359 - protein, 10 aa, synthetic construct
APFTKPSPFA

SEQ ID NO: 360 - protein, 10 aa, synthetic construct
ERTFTTYRMA

SEQ ID NO: 361 - protein, 10 aa, synthetic construct
TISRDGTRTY

SEQ ID NO: 362 - protein, 15 aa, synthetic construct
GSTDYPFLRPRDANY

SEQ ID NO: 363 - protein, 10 aa, synthetic construct
GRTFTTYRIA

SEQ ID NO: 364 - protein, 10 aa, synthetic construct
TISRDGTRTY

SEQ ID NO: 365 - protein, 15 aa, synthetic construct
GSTDYPFLRPRDANY

SEQ ID NO: 366 - protein, 10 aa, synthetic construct
GSGSSIVSMG

SEQ ID NO: 367 - protein, 9 aa, synthetic construct
SITSDGSRL

SEQ ID NO: 368 - protein, 6 aa, synthetic construct
LTPPTY

SEQ ID NO: 369 - protein, 10 aa, synthetic construct
GRTFTTYRIA

SEQ ID NO: 370 - protein, 10 aa, synthetic construct
TISRDGTRTY

SEQ ID NO: 371 - protein, 15 aa, synthetic construct
GSTDYPFLRPRDANY

SEQ ID NO: 372 - protein, 10 aa, synthetic construct
GRTFTTYRMG

SEQ ID NO: 373 - protein, 10 aa, synthetic construct
TISRDGTRPY

SEQ ID NO: 374 - protein, 15 aa, synthetic construct
RSTDYPFLRPRDANY

SEQ ID NO: 375 - protein, 10 aa, synthetic construct
GRTFTTYRMS

SEQ ID NO: 376 - protein, 10 aa, synthetic construct
TISPDGTRTY

SEQ ID NO: 377 - protein, 15 aa, synthetic construct
GSTVYPFLRPRDANY

SEQ ID NO: 378 - protein, 10 aa, synthetic construct
GRTFMTYRMG

SEQ ID NO: 379 - protein, 10 aa, synthetic construct
NISPDGTRTY

SEQ ID NO: 380 - protein, 15 aa, synthetic construct
GSTVYPFLRPRDANY

SEQ ID NO: 381 - protein, 10 aa, synthetic construct
TTTFSSYAMG

SEQ ID NO: 382 - protein, 10 aa, synthetic construct
TISGGGVYTY

SEQ ID NO: 383 - protein, 16 aa, synthetic construct
QRNIPRYGLESRNYDY

SEQ ID NO: 384 - protein, 10 aa, synthetic construct
GSVFRFYGMG

SEQ ID NO: 385 - protein, 9 aa, synthetic construct
DIRLSGRTN

SEQ ID NO: 386 - protein, 10 aa, synthetic construct
APFTIPSPFA

SEQ ID NO: 387 - protein, 11 aa, synthetic construct
SGSISSDNAMG

SEQ ID NO: 388 - protein, 9 aa, synthetic construct
SLTRDGSAI

SEQ ID NO: 389 - protein, 6 aa, synthetic construct
RTPPTY

SEQ ID NO: 390 - protein, 11 aa, synthetic construct
SGRTFSTYRMT

SEQ ID NO: 391 - protein, 10 aa, synthetic construct
TISRNGGSTY

SEQ ID NO: 392 - protein, 15 aa, synthetic construct
GSTAYPFLSPRDANY

SEQ ID NO: 393 - protein, 10 aa, synthetic construct
GRTFSTYRIT

SEQ ID NO: 394 - protein, 10 aa, synthetic construct
TISRSSSNPY

SEQ ID NO: 395 - protein, 15 aa, synthetic construct
GSTAYPFLSPRDANY

SEQ ID NO: 396 - protein, 10 aa, synthetic construct
GSVFRFYGMG

SEQ ID NO: 397 - protein, 9 aa, synthetic construct
DIRLSGRAD

SEQ ID NO: 398 - protein, 10 aa, synthetic construct
APFTIPSPFA

SEQ ID NO: 399 - protein, 11 aa, synthetic construct
GSGSSINTSMG

SEQ ID NO: 400 - protein, 9 aa, synthetic construct
SMPRGGGTI

SEQ ID NO: 401 - protein, 6 aa, synthetic construct
RTPPTY

SEQ ID NO: 402 - protein, 10 aa, synthetic construct
GSISSINAMG

SEQ ID NO: 403 - protein, 9 aa, synthetic construct
SMPRSGTAI

SEQ ID NO: 404 - protein, 6 aa, synthetic construct
HTPPTY

SEQ ID NO: 405 - protein, 11 aa, synthetic construct
GSGSSINTSMG

SEQ ID NO: 406 - protein, 9 aa, synthetic construct
SMPRGGGAI

SEQ ID NO: 407 - protein, 6 aa, synthetic construct
QTPPTY

SEQ ID NO: 408 - protein, 10 aa, synthetic construct
GSIFRFYGMG

SEQ ID NO: 409 - protein, 9 aa, synthetic construct
YIRLSGRTD

SEQ ID NO: 410 - protein, 10 aa, synthetic construct
PPFTKPSPFA

SEQ ID NO: 411 - protein, 10 aa, synthetic construct
GRTFSTYRMA

SEQ ID NO: 412 - protein, 10 aa, synthetic construct
TISPNGASTY

SEQ ID NO: 413 - protein, 15 aa, synthetic construct
GSTDYPFLRPRDANF

SEQ ID NO: 414 - protein, 10 aa, synthetic construct
GSIFRFYGMG

SEQ ID NO: 415 - protein, 9 aa, synthetic construct
DIRLSGRTD

SEQ ID NO: 416 - protein, 10 aa, synthetic construct
APFTKPSPFA

SEQ ID NO: 417 - protein, 10 aa, synthetic construct
GRTFTTYRMS

SEQ ID NO: 418 - protein, 10 aa, synthetic construct
TISPDGTRTY

SEQ ID NO: 419 - protein, 15 aa, synthetic construct
GSTVYPFLRPRDANY

SEQ ID NO: 420 - protein, 10 aa, synthetic construct
GRTFSTYRMG

SEQ ID NO: 421 - protein, 10 aa, synthetic construct
EISRNGGYTY

SEQ ID NO: 422 - protein, 15 aa, synthetic construct
GSTTYPFLSPRDANY

SEQ ID NO: 423 - protein, 10 aa, synthetic construct
GRTFTTYRMG

SEQ ID NO: 424 - protein, 10 aa, synthetic construct
TISRDGTRTY

SEQ ID NO: 425 - protein, 15 aa, synthetic construct
VSTDYPFLRPRDAAY

SEQ ID NO: 426 - protein, 9 aa, synthetic construct
GSSINTSMG

SEQ ID NO: 427 - protein, 9 aa, synthetic construct
SMPRGGGAL

SEQ ID NO: 428 - protein, 6 aa, synthetic construct
QTPPTF

SEQ ID NO: 429 - protein, 10 aa, synthetic construct
GRTFTTDRMA

SEQ ID NO: 430 - protein, 10 aa, synthetic construct
TISRDGTRPY

SEQ ID NO: 431 - protein, 15 aa, synthetic construct
GSTVYPFLRPRDANY

SEQ ID NO: 432 - protein, 10 aa, synthetic construct
EGTFSTYRMT

SEQ ID NO: 433 - protein, 10 aa, synthetic construct
TISRNGGSTY

SEQ ID NO: 434 - protein, 15 aa, synthetic construct
GSTAYPFLSPRDANY

SEQ ID NO: 435 - protein, 8 aa, synthetic construct
GRTFTTYR

SEQ ID NO: 436 - protein, 10 aa, synthetic construct
TVSRDGTRAY

SEQ ID NO: 437 - protein, 15 aa, synthetic construct
GSTDYPFLRPRDANY

SEQ ID NO: 438 - protein, 10 aa, synthetic construct
GRTFTTYRMS

SEQ ID NO: 439 - protein, 10 aa, synthetic construct
TISPDGTRTY

SEQ ID NO: 440 - protein, 15 aa, synthetic construct
GSTVYPFLRPRDANY

SEQ ID NO: 441 - protein, 10 aa, synthetic construct
GGTVTTYRMA

SEQ ID NO: 442 - protein, 10 aa, synthetic construct
TISRDGTSTY

SEQ ID NO: 443 - protein, 15 aa, synthetic construct
GSTTYPFLRPRDANY

SEQ ID NO: 444 - protein, 10 aa, synthetic construct
GSSLNIYAIG

SEQ ID NO: 445 - protein, 10 aa, synthetic construct
CISRSDGITF

SEQ ID NO: 446 - protein, 11 aa, synthetic construct
AGYRCALYMDY

SEQ ID NO: 447 - protein, 8 aa, synthetic construct
GRTFTTYR

SEQ ID NO: 448 - protein, 10 aa, synthetic construct
TVSRDGTRTY

SEQ ID NO: 449 - protein, 15 aa, synthetic construct
VSTDYPFLRPRDAAY

SEQ ID NO: 450 - protein, 10 aa, synthetic construct
GSISSNVSMG

SEQ ID NO: 451 - protein, 9 aa, synthetic construct
SMPRGGSAI

SEQ ID NO: 452 - protein, 6 aa, synthetic construct
RTPPTY

SEQ ID NO: 453 - protein, 10 aa, synthetic construct
GNIFRFYAMG

SEQ ID NO: 454 - protein, 10 aa, synthetic construct
DIRLSGSTNI

SEQ ID NO: 455 - protein, 10 aa, synthetic construct
APFTKPSPFA

SEQ ID NO: 456 - protein, 10 aa, synthetic construct
GRTFTTYRMG

SEQ ID NO: 457 - protein, 10 aa, synthetic construct
TISRDGTRTY

SEQ ID NO: 458 - protein, 15 aa, synthetic construct
LSTNYPFLRPRDANY

SEQ ID NO: 459 - protein, 11 aa, synthetic construct
GSGSSINTSMG

SEQ ID NO: 460 - protein, 9 aa, synthetic construct
SMPRGGGAI

SEQ ID NO: 461 - protein, 6 aa, synthetic construct
QTPPTY

SEQ ID NO: 462 - protein, 10 aa, synthetic construct
GRTFTTYRMG

SEQ ID NO: 463 - protein, 10 aa, synthetic construct
TISGDGTRPY

SEQ ID NO: 464 - protein, 15 aa, synthetic construct
GSTDYPFLRPRDANY

SEQ ID NO: 465 - protein, 10 aa, synthetic construct
GRTFTTYRMS

SEQ ID NO: 466 - protein, 10 aa, synthetic construct
TISPDGTRTY

SEQ ID NO: 467 - protein, 15 aa, synthetic construct
GSTVYPFLRPRDANY

SEQ ID NO: 468 - protein, 10 aa, synthetic construct
EGTFSTYRMT

SEQ ID NO: 469 - protein, 10 aa, synthetic construct
TISRNGGSTY

SEQ ID NO: 470 - protein, 15 aa, synthetic construct
GSTAYPFLSPRDANY

SEQ ID NO: 471 - protein, 365 aa, synthetic construct
MALPVTALLLPLALLLHAARPDVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 472 - protein, 257 aa, synthetic construct
DVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSSGGGGSGGGGSGGGGSDVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSS

SEQ ID NO: 473 - protein, 501 aa, synthetic construct
MALPVTALLLPLALLLHAARPDVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSSGGGGSGGGGSGGGGSDVQLQESGGGLVQAGGSLRLTCTTTFSSYAMGWFRQAPGKEREFVATISGGGVYTYYADSVKGRFTISRDNSKNTMYLQMNSLKFEDTAVYYCAQRNIPRYGLESRNYDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 474 - protein, 368 aa, synthetic construct
MALPVTALLLPLALLLHAARPDVQLQESGGGLVQAGGSLRLACAASGRTASSNAMGWFRQAPGKEREFVAAISGGGIYTDYADSVKGRFTISRDNSKNTMYLQMNSLKPEDTAVYYCAQRNIPRIGSGSRNYDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 475 - protein, 124 aa, synthetic construct; identical to SEQ ID NO: 208

SEQ ID NO: 476 - protein, 368 aa, synthetic construct
MALPVTALLLPLALLLHAARPDVQLQESGGGLVQAGGSLRLACAASGRTASSNAMGWFRQAPGKEREFVAAISGGGIYTDYADSVKGRFTISRDNSKNTMYLQMNSLKPEDTAVYYCAQRNIPRIGLGSGNYDYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 477 - protein, 241 aa, synthetic construct; identical to SEQ ID NO: 139

SEQ ID NO: 478 - protein, 485 aa, synthetic construct
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR